SEQUENCE LISTING (179 sequences)

SEQ ID NO: 1 - protein, 9 aa, Artificial Sequence
  Synthetic peptide
ILTEVLKGV

SEQ ID NO: 2 - protein, 9 aa, Artificial Sequence
  Synthetic peptide
WMGDRLMSL

SEQ ID NO: 3 - protein, 9 aa, Artificial Sequence
  Synthetic peptide
YMLVVGNIV

SEQ ID NO: 4 - protein, 9 aa, Artificial Sequence
  Synthetic peptide
LILVCLFCL

SEQ ID NO: 5 - protein, 9 aa, Artificial Sequence
  Synthetic peptide
KLANQINDL

SEQ ID NO: 6 - protein, 9 aa, Artificial Sequence
  Synthetic peptide
ALHSSVQSV

SEQ ID NO: 7 - protein, 9 aa, Artificial Sequence
  Synthetic peptide
LMPAVQNWL

SEQ ID NO: 8 - protein, 9 aa, Artificial Sequence
  Synthetic peptide
FQLQCDWNT

SEQ ID NO: 9 - protein, 9 aa, Artificial Sequence
  Synthetic peptide
RLWNSQSSI

SEQ ID NO: 10 - protein, 9 aa, Artificial Sequence
  Synthetic peptide
ILQNNEFGT

SEQ ID NO: 11 - protein, 9 aa, Artificial Sequence
  Synthetic peptide
ILVCLFCLL

SEQ ID NO: 12 - protein, 9 aa, Artificial Sequence
  Synthetic peptide
LLVRAREGV

SEQ ID NO: 13 - protein, 9 aa, Artificial Sequence
  Synthetic peptide
NLILILVCL

SEQ ID NO: 14 - protein, 9 aa, Artificial Sequence
  Synthetic peptide
NLTLDISKL

SEQ ID NO: 15 - protein, 9 aa, Artificial Sequence
  Synthetic peptide
TLIAVIMGL

SEQ ID NO: 16 - protein, 9 aa, Artificial Sequence
  Synthetic peptide
ILILVCLFC

SEQ ID NO: 17 - protein, 9 aa, Artificial Sequence
  Synthetic peptide
QINDLRQTV

SEQ ID NO: 18 - protein, 9 aa, Artificial Sequence
  Synthetic peptide
IINLILILV

SEQ ID NO: 19 - protein, 9 aa, Artificial Sequence
  Synthetic peptide
KLKEQIFEA

SEQ ID NO: 20 - protein, 9 aa, Artificial Sequence
  Synthetic peptide
RIWSGNQTL

SEQ ID NO: 21 - protein, 9 aa, Artificial Sequence
  Synthetic peptide
FIFTLIAVI

SEQ ID NO: 22 - protein, 9 aa, Artificial Sequence
  Synthetic peptide
ANLNPVTWV

SEQ ID NO: 23 - protein, 9 aa, Artificial Sequence
  Synthetic peptide
SLRPRVNYL

SEQ ID NO: 24 - protein, 9 aa, Artificial Sequence
  Synthetic peptide
VLNRSKRFI

SEQ ID NO: 25 - protein, 9 aa, Artificial Sequence
  Synthetic peptide
GVADGLANL

SEQ ID NO: 26 - protein, 9 aa, Artificial Sequence
  Synthetic peptide
AVIMGLIAV

SEQ ID NO: 27 - protein, 9 aa, Artificial Sequence
  Synthetic peptide
AVQNWLVEV

SEQ ID NO: 28 - protein, 9 aa, Artificial Sequence
  Synthetic peptide
NLVPGTEAI

SEQ ID NO: 29 - protein, 9 aa, Artificial Sequence
  Synthetic peptide
TIINLILIL

SEQ ID NO: 30 - protein, 9 aa, Artificial Sequence
  Synthetic peptide
AIAGVADGL

SEQ ID NO: 31 - protein, 9 aa, Artificial Sequence
  Synthetic peptide
HLQGREDNL

SEQ ID NO: 32 - protein, 9 aa, Artificial Sequence
  Synthetic peptide
VIMGLIAVT

SEQ ID NO: 33 - protein, 9 aa, Artificial Sequence
  Synthetic peptide
RLLSCIDST

SEQ ID NO: 34 - protein, 15 aa, Artificial Sequence
  Synthetic peptide
MVTPVTWMDNPIEIY

SEQ ID NO: 35 - protein, 15 aa, Artificial Sequence
  Synthetic peptide
VTWMDNPIEIYVNDS

SEQ ID NO: 36 - protein, 15 aa, Artificial Sequence
  Synthetic peptide
DNPIEIYVNDSVWVP

SEQ ID NO: 37 - protein, 15 aa, Artificial Sequence
  Synthetic peptide
EIYVNDSVWVPGPID

SEQ ID NO: 38 - protein, 15 aa, Artificial Sequence
  Synthetic peptide
NDSVWVPGPIDDRCP

SEQ ID NO: 39 - protein, 15 aa, Artificial Sequence
  Synthetic peptide
WVPGPIDDRCPAKPE

SEQ ID NO: 40 - protein, 15 aa, Artificial Sequence
  Synthetic peptide
PIDDRCPAKPEEEGM

SEQ ID NO: 41 - protein, 15 aa, Artificial Sequence
  Synthetic peptide
RCPAKPEEEGMMINI

SEQ ID NO: 42 - protein, 15 aa, Artificial Sequence
  Synthetic peptide
KPEEEGMMINISIGY

SEQ ID NO: 43 - protein, 15 aa, Artificial Sequence
  Synthetic peptide
EGMMINISIGYRYPP

SEQ ID NO: 44 - protein, 15 aa, Artificial Sequence
  Synthetic peptide
INISIGYRYPPICLG

SEQ ID NO: 45 - protein, 15 aa, Artificial Sequence
  Synthetic peptide
IGYRYPPICLGRAPG

SEQ ID NO: 46 - protein, 15 aa, Artificial Sequence
  Synthetic peptide
YPPICLGRAPGCLMP

SEQ ID NO: 47 - protein, 15 aa, Artificial Sequence
  Synthetic peptide
CLGRAPGCLMPAVQN

SEQ ID NO: 48 - protein, 15 aa, Artificial Sequence
  Synthetic peptide
APGCLMPAVQNWLVE

SEQ ID NO: 49 - protein, 15 aa, Artificial Sequence
  Synthetic peptide
LMPAVQNWLVEVPTV

SEQ ID NO: 50 - protein, 15 aa, Artificial Sequence
  Synthetic peptide
VQNWLVEVPTVSPIS

SEQ ID NO: 51 - protein, 15 aa, Artificial Sequence
  Synthetic peptide
LVEVPTVSPISRFTY

SEQ ID NO: 52 - protein, 15 aa, Artificial Sequence
  Synthetic peptide
PTVSPISRFTYHMVS

SEQ ID NO: 53 - protein, 15 aa, Artificial Sequence
  Synthetic peptide
PISRFTYHMVSGMSL

SEQ ID NO: 54 - protein, 15 aa, Artificial Sequence
  Synthetic peptide
FTYHMVSGMSLRPRV

SEQ ID NO: 55 - protein, 15 aa, Artificial Sequence
  Synthetic peptide
MVSGMSLRPRVNYLQ

SEQ ID NO: 56 - protein, 15 aa, Artificial Sequence
  Synthetic peptide
MSLRPRVNYLQDFSY

SEQ ID NO: 57 - protein, 15 aa, Artificial Sequence
  Synthetic peptide
PRVNYLQDFSYQRSL

SEQ ID NO: 58 - protein, 15 aa, Artificial Sequence
  Synthetic peptide
YLQDFSYQRSLKFRP

SEQ ID NO: 59 - protein, 15 aa, Artificial Sequence
  Synthetic peptide
FSYQRSLKFRPKGKP

SEQ ID NO: 60 - protein, 15 aa, Artificial Sequence
  Synthetic peptide
RSLKFRPKGKPCPKE

SEQ ID NO: 61 - protein, 15 aa, Artificial Sequence
  Synthetic peptide
FRPKGKPCPKEIPKE

SEQ ID NO: 62 - protein, 15 aa, Artificial Sequence
  Synthetic peptide
GKPCPKEIPKESKNT

SEQ ID NO: 63 - protein, 15 aa, Artificial Sequence
  Synthetic peptide
PKEIPKESKNTEVLV

SEQ ID NO: 64 - protein, 15 aa, Artificial Sequence
  Synthetic peptide
PKESKNTEVLVWEEC

SEQ ID NO: 65 - protein, 15 aa, Artificial Sequence
  Synthetic peptide
KNTEVLVWEECVANS

SEQ ID NO: 66 - protein, 15 aa, Artificial Sequence
  Synthetic peptide
VLVWEECVANSAVIL

SEQ ID NO: 67 - protein, 15 aa, Artificial Sequence
  Synthetic peptide
EECVANSAVILQNNE

SEQ ID NO: 68 - protein, 15 aa, Artificial Sequence
  Synthetic peptide
ANSAVILQNNEFGTI

SEQ ID NO: 69 - protein, 15 aa, Artificial Sequence
  Synthetic peptide
VILQNNEFGTIIDWA

SEQ ID NO: 70 - protein, 15 aa, Artificial Sequence
  Synthetic peptide
NNEFGTIIDWAPRGQ

SEQ ID NO: 71 - protein, 15 aa, Artificial Sequence
  Synthetic peptide
GTIIDWAPRGQFYHN

SEQ ID NO: 72 - protein, 15 aa, Artificial Sequence
  Synthetic peptide
DWAPRGQFYHNCSGQ

SEQ ID NO: 73 - protein, 15 aa, Artificial Sequence
  Synthetic peptide
RGQFYHNCSGQTQSC

SEQ ID NO: 74 - protein, 15 aa, Artificial Sequence
  Synthetic peptide
YHNCSGQTQSCPSAQ

SEQ ID NO: 75 - protein, 15 aa, Artificial Sequence
  Synthetic peptide
SGQTQSCPSAQVSPA

SEQ ID NO: 76 - protein, 15 aa, Artificial Sequence
  Synthetic peptide
QSCPSAQVSPAVDSD

SEQ ID NO: 77 - protein, 15 aa, Artificial Sequence
  Synthetic peptide
SAQVSPAVDSDLTES

SEQ ID NO: 78 - protein, 15 aa, Artificial Sequence
  Synthetic peptide
SPAVDSDLTESLDKH

SEQ ID NO: 79 - protein, 15 aa, Artificial Sequence
  Synthetic peptide
DSDLTESLDKHKHKK

SEQ ID NO: 80 - protein, 15 aa, Artificial Sequence
  Synthetic peptide
TESLDKHKHKKLQSF

SEQ ID NO: 81 - protein, 15 aa, Artificial Sequence
  Synthetic peptide
DKHKHKKLQSFYPWE

SEQ ID NO: 82 - protein, 15 aa, Artificial Sequence
  Synthetic peptide
HKKLQSFYPWEWGEK

SEQ ID NO: 83 - protein, 15 aa, Artificial Sequence
  Synthetic peptide
QSFYPWEWGEKRIST

SEQ ID NO: 84 - protein, 15 aa, Artificial Sequence
  Synthetic peptide
PWEWGEKRISTPRPK

SEQ ID NO: 85 - protein, 15 aa, Artificial Sequence
  Synthetic peptide
GEKRISTPRPKIVSP

SEQ ID NO: 86 - protein, 15 aa, Artificial Sequence
  Synthetic peptide
ISTPRPKIVSPVSGP

SEQ ID NO: 87 - protein, 15 aa, Artificial Sequence
  Synthetic peptide
RPKIVSPVSGPEHPE

SEQ ID NO: 88 - protein, 15 aa, Artificial Sequence
  Synthetic peptide
VSPVSGPEHPELWRL

SEQ ID NO: 89 - protein, 15 aa, Artificial Sequence
  Synthetic peptide
SGPEHPELWRLTVAS

SEQ ID NO: 90 - protein, 15 aa, Artificial Sequence
  Synthetic peptide
HPELWRLTVASHHIR

SEQ ID NO: 91 - protein, 15 aa, Artificial Sequence
  Synthetic peptide
WRLTVASHHIRIWSG

SEQ ID NO: 92 - protein, 15 aa, Artificial Sequence
  Synthetic peptide
VASHHIRIWSGNQTL

SEQ ID NO: 93 - protein, 15 aa, Artificial Sequence
  Synthetic peptide
HIRIWSGNQTLETRD

SEQ ID NO: 94 - protein, 15 aa, Artificial Sequence
  Synthetic peptide
WSGNQTLETRDCKPF

SEQ ID NO: 95 - protein, 15 aa, Artificial Sequence
  Synthetic peptide
QTLETRDCKPFYTID

SEQ ID NO: 96 - protein, 15 aa, Artificial Sequence
  Synthetic peptide
TRDCKPFYTIDLNSS

SEQ ID NO: 97 - protein, 15 aa, Artificial Sequence
  Synthetic peptide
KPFYTIDLNSSLTVP

SEQ ID NO: 98 - protein, 15 aa, Artificial Sequence
  Synthetic peptide
TIDLNSSLTVPLQSC

SEQ ID NO: 99 - protein, 15 aa, Artificial Sequence
  Synthetic peptide
NSSLTVPLQSCVKPP

SEQ ID NO: 100 - protein, 15 aa, Artificial Sequence
  Synthetic peptide
TVPLQSCVKPPYMLV

SEQ ID NO: 101 - protein, 15 aa, Artificial Sequence
  Synthetic peptide
QSCVKPPYMLVVGNI

SEQ ID NO: 102 - protein, 15 aa, Artificial Sequence
  Synthetic peptide
KPPYMLVVGNIVIKP

SEQ ID NO: 103 - protein, 15 aa, Artificial Sequence
  Synthetic peptide
MLVVGNIVIKPDSQT

SEQ ID NO: 104 - protein, 15 aa, Artificial Sequence
  Synthetic peptide
GNIVIKPDSQTITCE

SEQ ID NO: 105 - protein, 15 aa, Artificial Sequence
  Synthetic peptide
IKPDSQTITCENCRL

SEQ ID NO: 106 - protein, 15 aa, Artificial Sequence
  Synthetic peptide
SQTITCENCRLLSCI

SEQ ID NO: 107 - protein, 15 aa, Artificial Sequence
  Synthetic peptide
TCENCRLLSCIDSTF

SEQ ID NO: 108 - protein, 15 aa, Artificial Sequence
  Synthetic peptide
CRLLSCIDSTFNWQH

SEQ ID NO: 109 - protein, 15 aa, Artificial Sequence
  Synthetic peptide
SCIDSTFNWQHRILL

SEQ ID NO: 110 - protein, 15 aa, Artificial Sequence
  Synthetic peptide
STFNWQHRILLVRAR

SEQ ID NO: 111 - protein, 15 aa, Artificial Sequence
  Synthetic peptide
WQHRILLVRAREGVW

SEQ ID NO: 112 - protein, 15 aa, Artificial Sequence
  Synthetic peptide
ILLVRAREGVWIPVS

SEQ ID NO: 113 - protein, 15 aa, Artificial Sequence
  Synthetic peptide
RAREGVWIPVSMDRP

SEQ ID NO: 114 - protein, 15 aa, Artificial Sequence
  Synthetic peptide
GVWIPVSMDRPWEAS

SEQ ID NO: 115 - protein, 15 aa, Artificial Sequence
  Synthetic peptide
PVSMDRPWEASPSVH

SEQ ID NO: 116 - protein, 15 aa, Artificial Sequence
  Synthetic peptide
DRPWEASPSVHILTE

SEQ ID NO: 117 - protein, 15 aa, Artificial Sequence
  Synthetic peptide
EASPSVHILTEVLKG

SEQ ID NO: 118 - protein, 15 aa, Artificial Sequence
  Synthetic peptide
SVHILTEVLKGVLNR

SEQ ID NO: 119 - protein, 15 aa, Artificial Sequence
  Synthetic peptide
LTEVLKGVLNRSKRF

SEQ ID NO: 120 - protein, 15 aa, Artificial Sequence
  Synthetic peptide
LKGVLNRSKRFIFTL

SEQ ID NO: 121 - protein, 15 aa, Artificial Sequence
  Synthetic peptide
LNRSKRFIFTLIAVI

SEQ ID NO: 122 - protein, 15 aa, Artificial Sequence
  Synthetic peptide
KRFIFTLIAVIMGLI

SEQ ID NO: 123 - protein, 15 aa, Artificial Sequence
  Synthetic peptide
FTLIAVIMGLIAVTA

SEQ ID NO: 124 - protein, 15 aa, Artificial Sequence
  Synthetic peptide
AVIMGLIAVTATAAV

SEQ ID NO: 125 - protein, 15 aa, Artificial Sequence
  Synthetic peptide
GLIAVTATAAVAGVA

SEQ ID NO: 126 - protein, 15 aa, Artificial Sequence
  Synthetic peptide
VTATAAVAGVALHSS

SEQ ID NO: 127 - protein, 15 aa, Artificial Sequence
  Synthetic peptide
AAVAGVALHSSVQSV

SEQ ID NO: 128 - protein, 15 aa, Artificial Sequence
  Synthetic peptide
GVALHSSVQSVNFVN

SEQ ID NO: 129 - protein, 15 aa, Artificial Sequence
  Synthetic peptide
HSSVQSVNFVNDWQK

SEQ ID NO: 130 - protein, 15 aa, Artificial Sequence
  Synthetic peptide
QSVNFVNDWQKNSTR

SEQ ID NO: 131 - protein, 15 aa, Artificial Sequence
  Synthetic peptide
FVNDWQKNSTRLWNS

SEQ ID NO: 132 - protein, 15 aa, Artificial Sequence
  Synthetic peptide
WQKNSTRLWNSQSSI

SEQ ID NO: 133 - protein, 15 aa, Artificial Sequence
  Synthetic peptide
STRLWNSQSSIDQKL

SEQ ID NO: 134 - protein, 15 aa, Artificial Sequence
  Synthetic peptide
WNSQSSIDQKLANQI

SEQ ID NO: 135 - protein, 15 aa, Artificial Sequence
  Synthetic peptide
SSIDQKLANQINDLR

SEQ ID NO: 136 - protein, 15 aa, Artificial Sequence
  Synthetic peptide
QKLANQINDLRQTVI

SEQ ID NO: 137 - protein, 15 aa, Artificial Sequence
  Synthetic peptide
NQINDLRQTVIWMGD

SEQ ID NO: 138 - protein, 15 aa, Artificial Sequence
  Synthetic peptide
DLRQTVIWMGDRLMS

SEQ ID NO: 139 - protein, 15 aa, Artificial Sequence
  Synthetic peptide
TVIWMGDRLMSLEHR

SEQ ID NO: 140 - protein, 15 aa, Artificial Sequence
  Synthetic peptide
MGDRLMSLEHRFQLQ

SEQ ID NO: 141 - protein, 15 aa, Artificial Sequence
  Synthetic peptide
LMSLEHRFQLQCDWN

SEQ ID NO: 142 - protein, 15 aa, Artificial Sequence
  Synthetic peptide
EHRFQLQCDWNTSDF

SEQ ID NO: 143 - protein, 15 aa, Artificial Sequence
  Synthetic peptide
QLQCDWNTSDFCITP

SEQ ID NO: 144 - protein, 15 aa, Artificial Sequence
  Synthetic peptide
DWNTSDFCITPQIYN

SEQ ID NO: 145 - protein, 15 aa, Artificial Sequence
  Synthetic peptide
SDFCITPQIYNESEH

SEQ ID NO: 146 - protein, 15 aa, Artificial Sequence
  Synthetic peptide
ITPQIYNESEHHWDM

SEQ ID NO: 147 - protein, 15 aa, Artificial Sequence
  Synthetic peptide
IYNESEHHWDMVRRH

SEQ ID NO: 148 - protein, 15 aa, Artificial Sequence
  Synthetic peptide
SEHHWDMVRRHLQGR

SEQ ID NO: 149 - protein, 15 aa, Artificial Sequence
  Synthetic peptide
WDMVRRHLQGREDNL

SEQ ID NO: 150 - protein, 15 aa, Artificial Sequence
  Synthetic peptide
RRHLQGREDNLTLDI

SEQ ID NO: 151 - protein, 15 aa, Artificial Sequence
  Synthetic peptide
QGREDNLTLDISKLK

SEQ ID NO: 152 - protein, 15 aa, Artificial Sequence
  Synthetic peptide
DNLTLDISKLKEQIF

SEQ ID NO: 153 - protein, 15 aa, Artificial Sequence
  Synthetic peptide
LDISKLKEQIFEASK

SEQ ID NO: 154 - protein, 15 aa, Artificial Sequence
  Synthetic peptide
KLKEQIFEASKAHLN

SEQ ID NO: 155 - protein, 15 aa, Artificial Sequence
  Synthetic peptide
QIFEASKAHLNLVPG

SEQ ID NO: 156 - protein, 15 aa, Artificial Sequence
  Synthetic peptide
ASKAHLNLVPGTEAI

SEQ ID NO: 157 - protein, 15 aa, Artificial Sequence
  Synthetic peptide
HLNLVPGTEAIAGVA

SEQ ID NO: 158 - protein, 15 aa, Artificial Sequence
  Synthetic peptide
VPGTEAIAGVADGLA

SEQ ID NO: 159 - protein, 15 aa, Artificial Sequence
  Synthetic peptide
EAIAGVADGLANLNP

SEQ ID NO: 160 - protein, 15 aa, Artificial Sequence
  Synthetic peptide
GVADGLANLNPVTWV

SEQ ID NO: 161 - protein, 15 aa, Artificial Sequence
  Synthetic peptide
GLANLNPVTWVKTIG

SEQ ID NO: 162 - protein, 15 aa, Artificial Sequence
  Synthetic peptide
LNPVTWVKTIGSTTI

SEQ ID NO: 163 - protein, 15 aa, Artificial Sequence
  Synthetic peptide
TWVKTIGSTTIINLI

SEQ ID NO: 164 - protein, 15 aa, Artificial Sequence
  Synthetic peptide
TIGSTTIINLILILV

SEQ ID NO: 165 - protein, 15 aa, Artificial Sequence
  Synthetic peptide
TTIINLILILVCLFC

SEQ ID NO: 166 - protein, 15 aa, Artificial Sequence
  Synthetic peptide
NLILILVCLFCLLLV

SEQ ID NO: 167 - protein, 15 aa, Artificial Sequence
  Synthetic peptide
ILVCLFCLLLVCRCT

SEQ ID NO: 168 - protein, 15 aa, Artificial Sequence
  Synthetic peptide
LFCLLLVCRCTQQLR

SEQ ID NO: 169 - protein, 15 aa, Artificial Sequence
  Synthetic peptide
LLVCRCTQQLRRDSD

SEQ ID NO: 170 - protein, 15 aa, Artificial Sequence
  Synthetic peptide
RCTQQLRRDSDHRER

SEQ ID NO: 171 - protein, 15 aa, Artificial Sequence
  Synthetic peptide
QLRRDSDHRERAMMT

SEQ ID NO: 172 - protein, 15 aa, Artificial Sequence
  Synthetic peptide
DSDHRERAMMTMAVL

SEQ ID NO: 173 - protein, 15 aa, Artificial Sequence
  Synthetic peptide
RERAMMTMAVLSKRK

SEQ ID NO: 174 - protein, 15 aa, Artificial Sequence
  Synthetic peptide
MMTMAVLSKRKGGNV

SEQ ID NO: 175 - protein, 15 aa, Artificial Sequence
  Synthetic peptide
AVLSKRKGGNVGKSK

SEQ ID NO: 176 - protein, 15 aa, Artificial Sequence
  Synthetic peptide
KRKGGNVGKSKRDQI

SEQ ID NO: 177 - protein, 16 aa, Artificial Sequence
  Synthetic peptide
GNVGKSKRDQIVTVSV

SEQ ID NO: 178 - protein, 25 aa, Artificial Sequence
  Synthetic Peptide
IINLILILVCLFCLLLVCRCTQQLR

SEQ ID NO: 179 - protein, 27 aa, Artificial Sequence
  Synthetic Peptide
SQTITCENCRLLSCIDSTFNWQHRILL

(19) United States
(12) Patent Application Publication
(10) Pub. No.: US 2015/0250864 A1
(43) Pub. Date: Sep. 10, 2015

(54) ANTI-CANCER VACCINES

(71) Applicant: Board of Regents, The University of Texas System, Austin, TX (US)

(72) Inventor: Feng Wang-Johanning, Houston, TX (US)

(73) Assignee: Board of Regents, The University of Texas System, Austin, TX (US)

(21) Appl. No.: 14/410,025

(22) PCT Filed: Jun. 24, 2013
(86) PCT No.: PCT/US13/47353

§ 371 Date: Dec. 19, 2014

Related U.S. Application Data
(60) Provisional application No. 61/663,971, filed on Jun. 25, 2012.

(51) Int. Cl. A61K 39/00; G01N 33/574; A61K 39/12

(52) U.S. Cl. CPC A61K 39/0011; A61K 39/12; G01N 33/57415; G01N 33/57449; C12N 2740/10034; A61K 2039/585; G01N 2333/15; G01N 2333/57; G01N 2333/70539

(57) ABSTRACT

The present provides tumor-associated antigens and their use as vaccines for treating and limiting cancers in a patient. In specific aspects, HERV type K envelope proteins and peptides are provided. Such proteins and peptides can be used to elicit specific immune responses—both humoral and cellular—that target tumor cells expressing the HERV type K envelope protein.

This application claims benefit of priority to U.S. Ser. No. 61/663,971, filed Jun. 25, 2012, the entire content of which is hereby incorporated by references.

This invention was made with government support under grant no. W81XWH-07-1-0612 awarded by the National Institutes of Health. The government has certain rights in the invention.

### BACKGROUND OF THE INVENTION

1. Field of the Invention

The present invention relates generally to the fields of cancer and immunotherapy. More particularly, it concerns the identification of immunotherapeutic peptides and the development of peptide vaccines for the treatment and prevention of cancer.

2. Description of Related Art

Human endogenous retroviruses (HERVs) and elements containing long terminal repeat-like sequences may comprise up to 8% of the human genome. HERVs entered the human genome after fortuitous germ line integration of exogenous retroviruses and were subsequently fixed in the general population. They may have been preserved to ensure genome plasticity and this can provide the host with new functions, such as protection from exogenous viruses and fusiogenic activity (e.g., membrane fusion, exocytosis, or endocytosis). HERVs contain over 200 distinct groups and subgroups. The accumulation of mutations has led to a loss of infectivity of HERVs, and in general they are largely noninfectious retroviral remnants. However, open reading frames (ORFs) have been observed for ERV3, HERV-E 4-1, and HERV-K, but their significance is unknown.

The most biologically active HERVs are members of the HERV-K superfamily which is characterized by the presence of primer binding sites for lysine tRNA. Only HERV-K appears to have the full complement of open reading frames typical of replication competent mammalian retroviruses. The K family contains a central open reading frame (cORF) and is comparable to HIV-I Rev protein. HERV-K was originally identified by its homology to the mouse mammary tumor virus (MMTV), and is transcriptionally active in several human cancer tissues, including breast cancer tissues, as well as tumor cell lines, such as the human breast cancer cell line T47D and the teratocarcinoma cell line GH. HERV-K env mRNA is frequently expressed in human breast cancer and HERV-E mRNA is expressed in prostate cancer. Additionally, mRNA from multiple HERV families is transcribed only in ovarian cancer cell lines and tissues. For example, the expression of HERV-K env mRNA was greater in ovarian epithelial tumors than it was in normal ovarian tissues.

Breast cancer is a significant health problem for women in the United States and throughout the world. Although advances have been made in detection and treatment of the disease, breast cancer remains the second leading cause of cancer-related deaths in women, affecting more than 180,000 women in the United States each year. For women in North America, the life-time odds of getting breast cancer are now one in eight.

No vaccine or other universally successful method for the prevention or treatment of breast cancer is currently available. Management of the disease currently relies on a combination of early diagnosis (through routine screening procedures) and aggressive treatment, which may include one or more of a variety of treatments such as surgery, radiotherapy, chemotherapy, and hormone therapy. The course of treatment for a particular breast cancer is often selected based on a variety of prognostic parameters, including an analysis of specific-tumor markers. See, e.g., Porter-Jordan & Lippman (1994). However, the use of established markers often leads to a result that is difficult to interpret, and the high mortality observed in breast cancer patients indicates that improvements are needed in the treatment, diagnosis, and prevention of the disease.

Ovarian cancer is another leading cause of cancer deaths among women and has the highest mortality of any of the gynecologic cancers. Symptoms usually do not become apparent until the tumor compresses or invades adjacent structures, or ascites develops, or metastases become clinically evident. As a result, two thirds of women with ovarian cancer have advanced (Stage III or IV) disease at the time of diagnosis.

Potential screening tests for ovarian cancer include the bimanual pelvic examination, the Papanicolaou (Pap) smear, tumor markers, and ultrasound imaging. The pelvic examination, which can detect a variety of gynecologic disorders, is of unknown sensitivity in detecting ovarian cancer. Although pelvic examinations can occasionally detect ovarian cancer, small, early-stage ovarian tumors are often not detected by palpation due to the deep anatomic location of the ovary. Thus, ovarian cancers detected by pelvic examination are generally advanced and associated with poor survival. The pelvic examination may also produce false positives when benign adnexal masses (e.g., functional cysts) are found. The Pap smear may occasionally reveal malignant ovarian cells, but it is not considered to be a valid screening test for ovarian carcinoma. Ultrasound imaging has also been evaluated as a screening test for ovarian cancer, since it is able to estimate ovarian size, detect masses as small as 1 cm, and distinguish solid lesions from cysts.

Serum tumor markers are often elevated in women with ovarian cancer. Examples of these markers include carcinoembryonic antigen, ovarian cystadenocarcinoma antigen, lipid-associated sialic acid, NB/70K, TAG 72.3, CAI 15-3, and CA-125, respectively. Evidence is limited on whether tumor markers become elevated early enough in the natural history of occult ovarian cancer to provide adequate sensitivity for screening, and tumor markers may have limited specificity.

Tumor-associated antigens recognized by the immune system are a very attractive target for human cancer diagnostics and therapy. However, few immunotherapy approaches have been used for the treatment and prevention of cancers. One problem limiting the success of cancer vaccines is that the immune system generally does not recognize cancer cells as being foreign, which is a requirement for initiating an immune response. Cancer immunotherapy, however, is limited due in part to the limited number of tumor-associated antigens identified to date.

### SUMMARY OF THE INVENTION

Thus, in accordance with the present invention, there is provided a vaccine comprising a HERV-K enveloped (env) peptide or polypeptide comprising a sequence selected from SEQ ID NO: 1-179. The peptide or polypeptide may comprise 9 to 50 consecutive residues of HERV-K env, or 9 to 15 consecutive residues of HERV-K env. The peptide may be less than 50 residues in length, such as 9, 10, 11, 12, 13, 14, 15, 16, 17, 18, 19, 20, 25, 30, 35, 40, or 45 residues in length. The vaccine may further comprises an adjuvant, such as complete Freund's adjuvant, incomplete Freund's adjuvant, alum, Bacillus Calmette-Guerin, agonists and modifiers of adhesion molecules, tetanus toxoid, imiquinod, montanide, MPL, and QS21. The vaccine may further comprise an immunostimulatory cytokine.

The vaccine may comprise more than one peptide or polypeptide of HERV-K env. The peptide or polypeptide may be selected based on the antigenic profile of tumor to be treated, or on the HLA type of the patient. The peptide or polypeptide may comprise a T cell epitope, such as a T cell epitope is presented on the surface of an antigen presenting cell. The antigen presenting cell may be a dendritic cell used as a cellular vaccine to stimulate T cell immunity against the peptide, and thereby against the tumor. Alternatively, the peptide or polypeptide may comprise a B cell epitope.

In another embodiment, there is provided a method for treating a cancer in a patient comprising administering to said patient a therapeutically effective amount of a vaccine comprising HERV-K enveloped (env) peptide or polypeptide comprising a sequence selected from SEQ ID NO: 1-179. The method may comprise administering the vaccine more than once. The therapeutically effective amount may be in the range of 0.025 mg to 5.0 mg, or in the range of 0.025 mg to 1.0 mg. The cancer may be is a solid tumor, such as a bladder cancer, a lung cancer, a colon cancer, a prostate cancer, a liver cancer, a pancreatic cancer, a stomach cancer, a testicular cancer, a brain cancer, a lymphatic cancer, a skin cancer, a bone cancer, a soft tissue cancer. In particular, the cancer may be breast cancer or an ovarian cancer.

The peptide or polypeptide may comprise 9 to 50 consecutive residues of HERV-K env, or 9 to 15 consecutive residues of HERV-K env. The peptide may be less than 50 residues in length, such as 9, 10, 11, 12, 13, 14, 15, 16, 17, 18, 19, 20, 25, 30, 35, 40, or 45 residues in length. The vaccine may be administered systemically, including intravenously, intra-arterially, intra-peritoneally, intramuscularly, intradermally, intratumorally, orally, dermally, nasally, buccally, rectally, vaginally, by inhalation, or by topical administration. The vaccine may be administered locally, such as by direct intratumoral injection or injection into tumor vasculature.

The peptide or polypeptide may comprise a T cell epitope, such as a T cell epitope is pulsed or loaded on an antigen presenting cell. The antigen presenting cell may be a dendritic cell. The peptide or polypeptide may comprise a B cell epitope. The method may further comprise treating the patient with a second anticancer therapy, such as one administered simultaneously with the vaccine. Alternatively, the second anticancer therapy may be administered before or after the vaccine, or both before and after the vaccine. The second anticancer therapy may be a chemotherapeutic agent, radiotherapy, an immunotherapy, and hormonal therapy or a toxin therapy.

In yet another embodiment, there is provided a method for treating cancer in a patient comprising (a) contacting CTLs of said patient with a HERV-K enveloped (env) peptide or polypeptide comprising a sequence selected from SEQ ID NO: 1-33, 178 and 179; and (b) administering a therapeutically effective amount of the CTLs of step (b) to the patient. The method may further comprise expanding said CTL's by ex vivo or in vivo methods prior to administration. Contacting may comprise providing an antigen presenting cell loaded with said peptide or polypeptide or that expresses said peptide or polypeptide from an expression construct. The therapeutically effective amount of CTL cells required to provide therapeutic benefit may be from about 0.1×10⁵ to about 5×10⁷ cells per kilogram weight of the subject. The method may comprise performing step (b) more than once.

In still another embodiment, there is provided a method for treating a cancer in a patient comprising administering to said patient a therapeutically effective amount of a vaccine comprising an expression construct encoding a HERV-K enveloped (env) peptide or polypeptide comprising a sequence selected from SEQ ID NO: 1-179. The expression construct may a non-viral expression construct, or a viral expression construct. The expression construct may be located in an antigen presenting cell. The method may comprise administering the vaccine more than once.

In still yet another embodiment, there is provided a method of identifying a subject with breast and ovarian cancer comprising determining the presence or amount of an anti-HERV-K envelope protein immune response in a sample from said subject. The immune response may be a humoral immune response, and may be evaluated using ELISA. The immune response may be a cellular immune response, and may be evaluated using an IFN-γ stimulation assay, and may be evaluated following depletion of T regulatory cells. The sample may be peripheral blood, serum, plasma, or a tumor biopsy. The method may further comprise assessing the presence or amount of an anti-HERV-K envelope protein immune response in a sample from said subject a second time. The method may also further comprising positively correlating a reduced immune response over time with therapeutic response, and may even further comprise treating the patient to produce a therapeutic response.

In a further embodiment, there is provided a method for stimulating an anti-cancer B cell response in a cancer patient comprising (a) contacting B cells of said patient with a HERV-K enveloped (env) peptide or polypeptide comprising a sequence selected from SEQ ID NO: 34-177; and (b) administering a therapeutically effective amount of the B cells of step (a) to the patient. The method may further comprise stimulating said B cells ex vivo or in vivo methods prior to administration. Contacting may comprise providing an antigen presenting cell loaded with said peptide or polypeptide or that expresses said peptide or polypeptide from an expression construct. The therapeutically effective amount of B cells required to provide therapeutic benefit ma be from about 0.1×10⁵ to about 5×10⁷ cells per kilogram weight of the subject. The method may comprise performing step (b) more than once.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. T-cell proliferation. (FIG. 1A) Detection of HERV-K-specific T cell responses (dendritic cells pulsed with HERV-K cRNA) in ovarian cancer patient PBMC (OC 1 to OC 4), compared with normal female donors (NL1 to NL3). Higher HERV-K-specific T cell responses were found in PBMC and IVS cells obtained from ovarian cancer patients. (FIG. 1B) Higher HERV-K-specific T cell responses were found in PBMC and IVS cells obtained from ovarian cancer patients (OC 60 and OC 44) than from a patient with teratoma (OC 63). (FIG. 1C) T cell proliferation was compared between patients with benign diseases (bDC+KSU; N=5) and ovarian cancer (tDC+KSU; N=9). Dendritic cells pulsed with KLH were used for controls and were compared with DC pulsed with HERV-K surface (SU) protein.

FIGS. 2A-D. IFN-γ ELISPOT was used to determine HERV-K-specific T cell response. (FIG. 2A) More spots (red box) were detected in IVS cells (DC+KSU) from an ovarian cancer patient with moderately to poorly differentiated adenocarcinoma, in comparison to three normal female donors (ND263559, 273010, and 273011; black boxes) stimulated with a single HERV-K SU protein. No spots were detected in controls using keyhole limpet hemocyanin (KLH) as antigen. (FIG. 2B) More spots (red box for KSU and green box for KTM) were detected in IVS cells (DC+KSU) from an ovarian cancer patient (Acc 153) with high grade papillary serous carcinoma before surgery (0 month) or after surgery (6 months), compared to one normal female donor (ND 273011) stimulated one time with HERV-K SU protein. IVS cells were generated from PBMC stimulated with a single HERV-K SU or TM antigen. No spot was detected in controls using KLH as antigen. (FIG. 2C) IFN-γ spots were detected in IVS cells (1×10⁵ cells loaded) from cancer patients (#816463 and #618329: blood collected 6 months after surgery) but not in a breast cancer patient with invasive ductal carcinoma (#814349, collected 6 months after surgery) or a patient (717335) with a mass (no malignancy identified), stimulated one time with either HERV-K SU (surface domain of env protein) or TM (transmembrane domain) protein. DC pulsed with KLH served as control. (FIG. 2D) A summary of IFN-γ ELISPOT results from ovarian cancer (N=10) vs. patients with benign disease (N=10) is shown here.

FIGS. 3A-D. A CTL assay was employed to determine HERV-K-specific T cell cytotoxicity toward their own tumor cells or matched normal epithelial cells. HERV-K-specific T cells were generated from a patient diagnosed with malignant mixed mullerian tumor (115, FIG. 3A), high grade serous carcinoma (104, FIG. 3B), or metastatic adenocarcinoma (122, FIG. 3C). Primary tumor cells (red color) and matched uninvolved epithelial cells (green color) cultured from biopsy tissues obtained at surgery from the same patient were used as target cells. HERV-K-specific T cells were cytotoxic only toward their own ovarian tumor epithelial cells, but not toward their matched uninvolved ovarian cells. HPV16 E6 or KLH was used as control. (FIG. 3D) A summary of CTL results for ovarian cancer (N=15) vs. benign patients (N=11) is shown here, as well as a summary of CTL against ovarian cancer tumor cells vs. uninvolved cells. Abbreviations: Both=no Treg depleted, Treg−=T reg depleted, Treg+=T reg cells.

FIGS. 4A-C. (FIG. 4A) A summary of CTL data in ovarian cancer patients vs. patients with benign disease. Significantly higher percentages of HERV-K specific lysis were observed in patients with ovarian cancer, compared to patients with benign diseases (P<0.0001, unpaired t-test). (FIG. 4B) Significantly higher percentages of HERV-K specific lysis were observed in tumor cells compared with matched uninvolved ovarian epithelial cells obtained from the same ovarian cancer patients (P=0.0006, paired t-test). (FIG. 4C) Significantly higher percentages of HERV-K specific lysis were observed in IVS cells without regulatory T cells (T-reg, P=0.0007), or with T-reg (T-reg+, P=0.0006), compared with IVS (both).

FIG. 5. An ELISA assay was employed to determine the B cell epitopes of 15-mer peptide pools (144 peptides in 12 small pools (SP)). Multiple epitopes were discovered from 6H5 mAb (6H5 lot 14) in peptide pools including SP2, SP4, SP5, SP7, SP8, SP9, SP11, and SP12. Each small pool contains twelve 15-mer peptides.

FIG. 6. Individual epitopes were further determined by ELISA using single peptides with various monoclonal antibodies (4E6, 4E11, 6H5, 6E11, and 5° F.11). Peptides with higher optical density (OD) values (greater than 1.0 OD405 nm) were considered to be B cell epitopes.

FIG. 7. Several novel epitopes were detected in BC patients by ELISA in SP8 and SP9, including 94, 108, 91, 88, 89, and 95 from pool 9.

FIGS. 8A-B. Several epitopes were found by ELISA in SP8 or SP9 using patient sera (FIG. 12B), such as 94, 108, 91, 88, 89, and 95. However, 108 is the only active epitope in each patient's serum.

FIGS. 9A-B. Biacore was employed for epitope mapping. 6E11 mAb had binding epitopes different from 4E11 or 4E6 mAbs. Binding epitopes were not different between 4E6 and 4E11.

FIGS. 10A-B. Immune response was determined in mice immunized with candidate MAPs. (FIG. 10A) ELISA assay was used to determine B cell MAPs (#82-83). (FIG. 10B) ELISPOT assay was used for T cell MAPs (#82-83).

FIGS. 11A-B. ELISA was employed to screen the candidate MAPs. Sera obtained from tumor in situ (TIS) (N=20) or normal female controls (N=10) were compared.

FIGS. 12A-B. Identification of HLA-A2 T-cell specific peptides using anti-human IFN-γ ELISPOT with T cells from one HLA A2⁺ normal donor. (FIG. 12A) T cells, stimulated by peptide pool-pulsed homologous B cells three (3^{rd} T cells) or four (4^{th} T cells) times were reactive with two pools from a HERV-K HLA A2⁺9-mer peptide library (pool #1-11 and pool #12-22). NP is no peptide control. (FIG. 12B) Photographs of actual plates for FIG. 12A.

FIGS. 13A-B. Identification of HLA-A2 T cell specific epitopes using anti-human IFN-γ ELISPOT. T cells obtained from PBMC stimulated by peptide pool-pulsed homologous B cells three (3^{rd} T cells) or four (4^{th} T cells) times, were reactive with individual peptides. (FIG. 13A) Image of ELISPOT. (FIG. 13B) The code of peptides evaluated in ELISPOT assays.

FIGS. 14A-C. Summary of number of ELISPOTs from each individual peptide. Numbers of spots detected (pool 1-11, pool 12-22, and pool 23-33) in ELISPOTS with a single peptide are shown. NP contains no peptide (as control). (FIG. 14A) Summary of number of ELISPOTs from each individual peptide including #1, #2, #3, #5, #6, #7, #8, and #9, and peptide pool #1 to #11. (FIG. 14B) Summary of number of ELISPOTs from each individual peptide including #12, #13, #14, #15, #17, #18, #19, #20, #22 and peptide pool #12 to #22. (FIG. 14C) Summary of number of ELISPOTs from each individual peptide including #23, #24, #25, #26, #27, #28, #30, #31, #32, #33, and peptide pool #23 to #33. 4^{th} T cells indicated the T cells had been stimulated with peptide pool 4 times and 3^{rd} T cells indicated the T cells had been stimulated with peptide pool 3 times.

FIGS. 15A-C. ELISPOT was employed to determine the production of IFN-γ in twelve 15-mer small pools. (FIG. 15A) Numbers of spots detected in multiple pools are shown for a breast cancer patient diagnosed with IDC (Acc 30) or a patient diagnosed with teratoma (Acc 32). (FIG. 15B) Numbers of spots detected in multiple pools are shown for a patient diagnosed with left breast minute foci of sugg papillary lesion (967092) or IDC (Acc 31). SP 1-12 represents small peptide pools #1-12. Pool 0 contains no peptides (as control). (FIG. 15C) T cells obtained from PBMC stimulated once with HERV-K-specific pulsed homologous B cells were reactive with individual MAPs, pools of 9-mer peptides, or single 9-mer peptides. NP: no peptide control.

FIGS. 16A-D. Identification of T epitopes by IFN-γ ELISPOT. TIL were obtained from melanoma patient specimens, and ELISPOT was used to determine epitopes. (FIG. 16A) Analysis of IFN-γ spots. SP7 gave the largest number of spots, and peptide #73 from SP 7 similarly gave a large number of spots. (FIG. 16B) Images of ELISPOT results using peptide pools. Columns 1 to 3 are triplicate analyses of wells coated with NP (row A), SP1 (row B), SP2 (row C), SP3 (row D), SP4 (row E), SP5 (row F), SP6 (row G), and PMA/Ionomycin (row H). Columns 4 to 6 are triplicate analyses of wells coated with SP7 (row A), SP8 (row B), SP9 (row C), SP10 (row D), SP11 (row E), SP12 (row F) and pools 1-23/30-51 (row G). (FIG. 16C) Images of ELISPOT results using single peptides. Columns 7 to 9 are triplicate analyses of wells coated with SP7, peptide #73 (row A), SP7#74 (row B), SP7#75 (row C), SP7#76 (row D), SP7#77 (row E), and SP7#78 (row F). Columns 10 to 12 are triplicate analyses of SP7#79 (row A), SP7#80 (row B), SP7#81 (row C), SP7#82 (row D), and SP7#83 (row E). (FIG. 16D) Repeat of the experiment described in FIG. 16B. 1-23/30-51: EBV and flu virus positive control peptides (yellow box). PMA/Ionomycin (green box) was used as mitogen (unspecific antigen) to stimulate the T cells to produce cytokines (e.g., IFN-γ). This positive control demonstrated that the T cells were active in this assay.

FIG. 17. T cell stimulation by HERV-K peptides. Peptides (p73-76 and p18-135) or HERV-K SU fusion proteins were used to stimulate PBMC and evaluated in vitro for their ability to stimulate T cells in PBMCs from healthy subjects (ND1007083; left) and/or cancer patients (BC 243; right), based on interferon-γ(IFN-γ) release by ELISA. KLH peptide and GST protein were used as controls. Other negative controls included media only, DMSO, and PBS.

FIG. 18. T cell stimulation by HERV-K peptides. Peptides (p73-76: left; and p18-135: middle) or HERV-K SU fusion proteins (right) were used to stimulate PBMC and evaluated in vitro for their ability to stimulate T cells in PBMCs from healthy subjects and/or cancer patients (BC 243; middle), based on interferon-γ (IFN-γ) release by ELISA. OD values of interferon-γ (IFN-γ) release were compared between normal donors (ND447549, ND1001366, and ND1007083) and cancer patients (OC222, OC 210, OC212, and BC243). OC is ovarian cancer and BC is breast cancer

FIG. 19. T cell stimulation by HERV-K peptides. Peptides (p73-76 and p18-135) or HERV-K SU fusion proteins were used to stimulate PBMC and evaluated in vitro for their ability to stimulate T cells in PBMCs from healthy subjects and/or cancer patients, based on Granzyme B (Top panel) and interlukin-2 (IL-2; bottom panel) release by ELISA. OD values of Granzyme B or IL-2 release were compared between normal donors (ND427478, ND329966, and ND341277) and cancer patients (BC243 and OC212).

FIG. 20. CD3+ and CD8+ T by HERV-K peptides. CD3+ and CD8+ T cells obtained from PBMC of patient OC213, pulsed with HERV-K SU protein, were sorted by flow cytometry and individual CD3+ and CD8+ T cells were used for further T expansion. CD3+ and CD8+ T cells obtained from patient OC213 had 63.3% CD3+ cells and 8.44% CD8+ cells.

FIGS. 21A-C. T Cell Stimulation. 9 mer Peptides (HERVK-3, HERVK-4, HERVK-11, and HERVK-18; see Table 1), p73-76 and p18-135 (Table 1 178 and 179), or HERV-K SU fusion proteins were used to stimulate PBMC and evaluated in vitro for their ability to stimulate T cells in PBMCs from healthy subjects and/or cancer patients, based on IFN-γ and Granzyme B release by ELISA. (FIG. 21A) OD values of IFN-γ release were compared in a normal donor (ND291812). (FIG. 21B) OD values of IFN-γ release were compared between a normal donor (ND291817) and an ovarian cancer patient (OC153). (FIG. 21C) OD values of Granzyme B release were compared between a normal donor (ND291817) and an ovarian cancer patient (OC153). Each dot represents an individual clone. Negative controls measured release of effector molecules from cancer cells after treatment with supernatants obtained from PBMCs without stimulation with HERV-K peptides or protein; positive controls were stimulated with PMA/ONO.

FIG. 22. CTL assay. A CTL assay was employed to determine HERV-K-specific CAR T cell cytotoxicity toward breast cancer cells transduced with shRNA targeting HERV-K env RNA (shRNA) or matched scrambled control (cont). The scFv sequence of the HERV-K monoclonal antibody (6H5) was used to construct a CAR. PBMCs obtained from BC108 (metastatic IDC), OC153 (high grade papillary serous carcinoma), and two normal donors were electroporated with HERV—K-CAR, then propagated on aAPC (HERV-K+K562 cells) in the presence of IL2 and IL21. Higher lysis was demonstrated in breast cancer cell lines (MDA-MB-231 and SKBr3) transduced with control shRNA, compared with breast cancer cell lines transduced with HERV-K shRNA. The data demonstrate specific killing in an antigen specific manner.

### DESCRIPTION OF ILLUSTRATIVE EMBODIMENTS

The identification of tumor-associated antigens (TAAs) is an approach for generating effective cancer vaccines that has received increased emphasis, and is viewed as a very promising method for cancer therapy. This approach relies on utilizing the immune system to eradicate malignant cells in response to the TAA. Optimally, the best tumor antigens for immunotherapy are those that are highly tumor specific and against which little or no immune tolerance exists. More importantly, these viral proteins are able to induce both antibody and T cell responses in vivo and in vitro.

A large number of TAAs and TAA-derived epitopes have been identified, and some of these proteins and peptide derivatives already in clinical vaccine trials. Even though results in the use of TAAs as cancer vaccines have been positive, these types of cancer vaccine trials have only shown partial success because tumor cells may have escaped surveillance by the immune system through loss and/or down-regulation of TAAs. One of the main hurdles encountered is the need to overcome self-tolerance mechanisms that limit the immune response against these types of tumor antigens Immunogenic TAAs that elicit minimal immune escape therefore represent the most optimal vaccine candidates for immunotherapy of cancer. Viral antigens may be better suited to trigger stronger antitumor T-cell responses due to their foreign nature.

Although only a few human cancers are associated with a viral etiology, recent evidence indicates that endogenous retroviruses silenced under normal conditions throughout our lifetime are induced in cancer cells and may be a new source of viral-like tumor antigens. The human genome harbors many endogenous retrovirus (ERV) sequences and some of them may continue to perform various retroviral functions, including tumor induction. There has been increasing appreciation of the association between the expression of human endogenous retrovirus type K (HERV-K) env gene and breast cancer and other cancers. The inventor reported that HERV-K env RNA and protein can trigger immune response in only breast cancer patients (Wang-Johanning et al., 2008), a finding which supports the concept that HERV-K may be an excellent TAA.

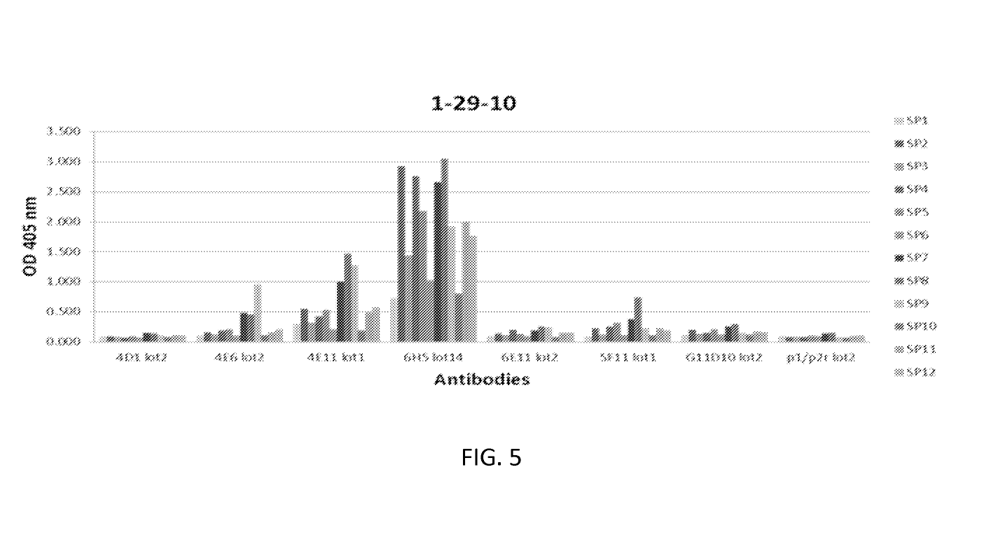

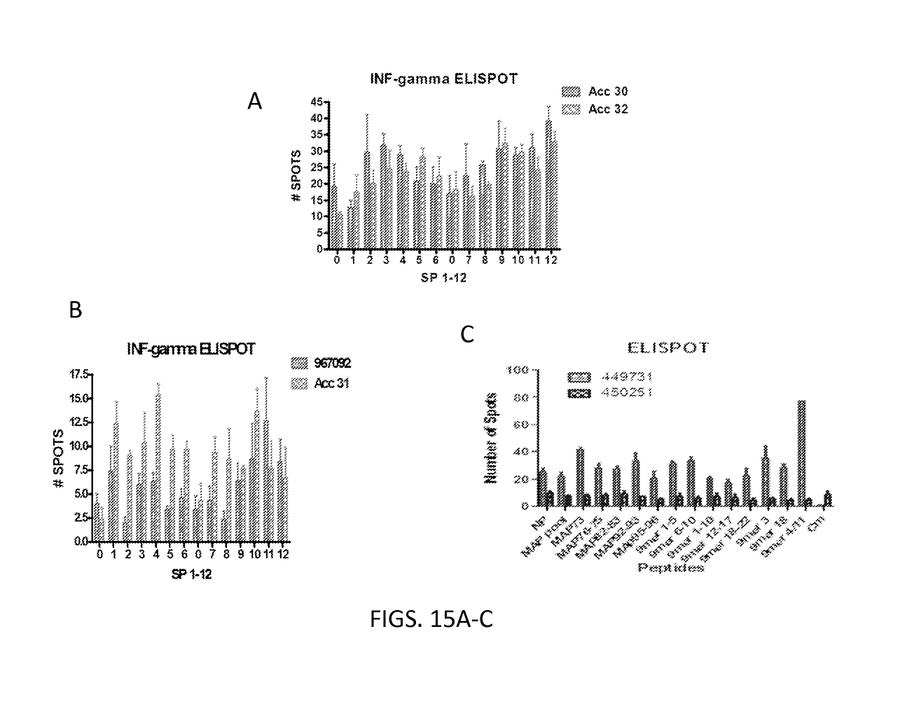

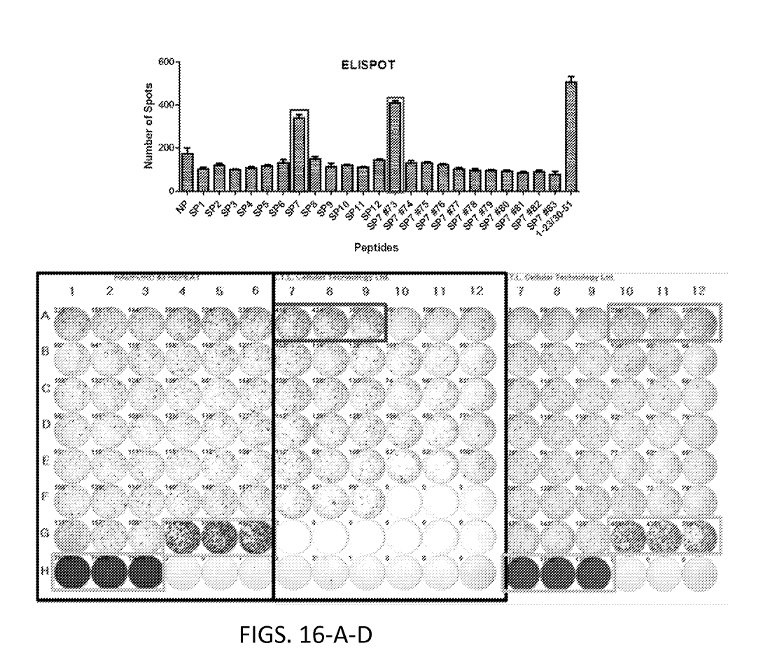

Based on the work described herein, HERV-K or other HERV family env proteins may in fact be the best tumor antigens for immunotherapy based on the present data. In addition, 15-mer (144 peptides) of HERV-K env protein have been designed, synthesized and tested by ELISA (FIGS. 5-8). A peptide library has been designed and specific epitopes of HERV-K env protein for CD4 T cells have been identified. T cell epitopes in small pool #7 (SP7), which contains 12 peptides, were identified by tumor-infiltrating lymphocytes (TIL) obtained from a melanoma patient using IFN-γ ELISPOT. A single peptide epitope from the SP7 pool was further identified using this same assay (FIG. 16). An ELISA assay has been employed to identify B cell epitopes from 144 peptides using anti-HERV-K monoclonal antibody or cancer patient sera. Sera from normal female donors or females with benign disease were used as control. In addition, a peptide library of nine amino acids (33 peptides) containing putative binding motifs for HLA-A0201 molecules has been selected and synthesized. HLA-A2⁺ PBMCs from breast cancer patients or normal donors are being used to determine the candidate epitopes by IFN-γ ELISPOT (FIGS. 15A-C). In addition, infectious HERV viruses were demonstrated in tumor cells (breast cancer, ovarian cancer, pancreatic and melanoma patients), but not in uninvolved epithelial cells or benign epithelial cells. Higher reverse transcriptase activity was demonstrated in cancer patient blood samples as well as tumor biopsies, but not in normal or benign control donors. The presence of these endogenous retroviral mRNAs, proteins and viral particles was further demonstrated using additional assays.

These and other aspects of the present disclosure are described in detail below.

### I. DEFINITIONS

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Major histocompatibility complex” or “MHC” is a cluster of genes that plays a role in control of the cellular interactions responsible for physiologic immune responses. In humans, the MHC complex is also known as the HLA complex. For a detailed description of the MHC and HLA complexes (see Paul, 1993).

“Human leukocyte antigen” or “HLA” is a human class I or class II major histocompatibility complex (MHC) protein.

An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, HLA family, and HLA xx-like supertype molecules (where xx denotes a particular HLA type), are synonyms.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “supermotif” is a peptide binding specificity shared by HLA molecules encoded by two or more HLA alleles. Thus, a preferably is recognized with high or intermediate affinity (as defined herein) by two or more HLA antigens.

“Cross-reactive binding” indicates that a peptide is bound by more than one HLA molecule; a synonym is degenerate binding.

A “protective immune response” refers to a CTL and/or an HTL response to an antigen derived from an infectious agent or a tumor antigen, which prevents or at least partially arrests disease symptoms or progression. The immune response may also include an antibody response which has been facilitated by the stimulation of helper T cells.

### II. HERV K AND ENVELOPE PROTEIN

Human endogenous retroviruses (HERVs) comprise approximately 8% of the human genome. Most of the identified elements are defective due to mutations and/or deletions within their genes, but some elements have conserved full-length open reading frames. A systematic search has led to the identification of 18 coding env genes. The most important contributor to this list is the HERV-K(HML2) family, with six coding env genes. Among the other HERV families, it has previously been demonstrated that two env genes, from the HERV-W and HERV-FRD families, are indeed functional since they can induce cell-cell fusion when expressed in cells possessing the corresponding receptors and since they render lentiviral particles infectious in pseudotype experiments.

The proteins encoded by the six complete HERV-K env genes are highly conserved, with more than 97% identity at the amino acid level, consistent with their recent integration into the human genome. The structural organization of the HERV-K ENVs is canonical, with a signal peptide at the N-terminal end (albeit longer than usual), an RX(K/R)R consensus cleavage site for the cellular furin protease that splits the surface (SU) and TM subunits, a hydrophobic fusion domain at the N-terminal end of the TM subunit, the two conserved cysteine residues in the ectodomain of the TM subunit, and a hydrophobic transmembrane anchor domain.

### III. PEPTIDES AND POLYPEPTIDE ANTIGENS

Computer programs are available to assist with predicting antigenic portions and epitopic core regions of proteins. Examples include those programs based upon the Jameson-Wolf analysis (Jameson and Wolf, 1988; Wolf et al., 1988), the program PepPlot® (Brutlag et al., 1990; Weinberger et al., 1985), and other new programs for protein tertiary structure prediction (Fetrow and Bryant, 1993). Another commercially available software program capable of carrying out such analyses is MacVector (IBI, New Haven, Conn.). U.S. Pat. No. 4,554,101, incorporated herein by reference, teaches the identification and preparation of epitopes from Primary amino acid sequences on the basis of hydrophilicity. Through the methods disclosed in Hopp, one of skill in the art would be able to identify epitopes from within an amino acid sequence such as the tumor-associated HLA-restricted peptides sequences disclosed herein. Numerous scientific publications have also been devoted to the prediction of secondary structure, and to the identification of epitopes, from analyses of amino acid sequences (Chou and Fasman, 1974a, b; 1978a, b; 1979). Any of these may be used, if desired, to supplement the teachings of Hopp in U.S. Pat. No. 4,554,101.

In other methods, major antigenic determinants of a tumor-associated peptide or polypeptide may be identified by an empirical approach in which portions of the gene encoding the tumor-associated peptides or polypeptides are expressed in a recombinant host, and the resulting proteins tested for their ability to elicit an immune response. For example, PCR™ can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

The present invention has employed an empirical approach to identifying antigenic tumor-associated peptides and polypeptides for use as vaccines. As used herein, an “antigenic composition” may comprise an antigen (e.g., a peptide or polypepide), a nucleic acid encoding an antigen (e.g., an antigen expression vector), or a cell expressing or presenting an antigen. For an antigenic composition, such as a tumor-associated HLA-restricted peptide or antigen of the present invention, to be useful as a vaccine, the antigenic composition must induce an immune response to the antigen in a cell, tissue or animal (e.g., a human). In particular embodiments, the antigenic composition comprises or encodes all or part of the sequences shown in SEQ ID NO: 1-179, or an immunologically functional equivalent thereof

TABLE 1-9
9-MER T CELL EPITOPES

| PEPTIDE | | POSITION | SEQ |
|---|---|---|---|
| NAME | SEQUENCE | START | ID NO |
| HERVK-1 | ILTEVLKGV | 340 | 1 |
| HERVK-2 | WMGDRLMSL | 424 | 2 |
| HERVK-3 | YMLVVGNIV | 276 | 3 |
| HERVK-4 | LILVCLFCL | 532 | 4 |
| HERVK-5 | KLANQINDL | 410 | 5 |
| HERVK-6 | ALHSSVQSV | 379 | 6 |
| HERVK-7 | LMPAVQNWL | 61 | 7 |
| HERVK-8 | FQLQCDWNT | 436 | 8 |
| HERVK-9 | RLWNSQSSI | 399 | 9 |
| HERVK-10 | ILQNNEFGT | 142 | 10 |
| HERVK-11 | ILVCLFCLL | 533 | 11 |
| HERVK-12 | LLVRAREGV | 314 | 12 |
| HERVK-13 | NLILILVCL | 529 | 13 |
| HERVK-14 | NLTLDISKL | 474 | 14 |
| HERVK-15 | TLIAVIMGL | 358 | 15 |
| HERVK-16 | ILILVCLFC | 531 | 16 |
| HERVK-17 | QINDLRQTV | 414 | 17 |
| HERVK-18 | IINLILILV | 527 | 18 |
| HERVK-19 | KLKEQIFEA | 481 | 19 |
| HERVK-20 | RIWSGNQTL | 239 | 20 |
| HERVK-21 | FIFTLIAVI | 355 | 21 |
| HERVK-22 | ANLNPVTWV | 511 | 22 |
| HERVK-23 | SLRPRVNYL | 90 | 23 |
| HERVK-24 | VLNRSKRFI | 348 | 24 |
| HERVK-25 | GVADGLANL | 505 | 25 |
| HERVK-26 | AVIMGLIAV | 361 | 26 |
| HERVK-27 | AVQNWLVEV | 64 | 27 |
| HERVK-28 | NLVPGTEAI | 495 | 28 |
| HERVK-29 | TIINLILIL | 526 | 29 |
| HERVK-30 | AIAGVADGL | 502 | 30 |
| HERVK-31 | HLQGREDNL | 414 | 31 |
| HERVK-32 | VIMGLIAVT | 362 | 32 |
| HERVK-33 | RLLSCIDST | 298 | 33 |
| P73-76 | IINLILILVCLFCLLLV | 289 | 178 |
| | CRCTQQLR | | |
| P18-135 | SQTITCENCRLLSCIDS | 527 | 179 |
| | TFNWQHRILL | | |

TABLE 2
15-MER CD4 T AND B CELL EPITOPES (SORTED BY POOL)

| Reference | | |
|---|---|---|
| number | | |
| | | SP1: (LP1) |
| 1 | MVTPVTWMDNPIEIY | SEQ ID NO: 34 |
| 2 | VTWMDNPIEIYVNDS | SEQ ID NO: 35 |
| 3 | DNPIEIYVNDSVWVP | SEQ ID NO: 36 |
| 4 | EIYVNDSVWVPGPID | SEQ ID NO: 37 |
| 5 | NDSVWVPGPIDDRCP | SEQ ID NO: 38 |
| 6 | WVPGPIDDRCPAKPE | SEQ ID NO: 39 |
| 7 | PIDDRCPAKPEEEGM | SEQ ID NO: 40 |
| 8 | RCPAKPEEEGMMINI | SEQ ID NO: 41 |
| 9 | KPEEEGMMINISIGY | SEQ ID NO: 42 |
| 10 | EGMMINISIGYRYPP | SEQ ID NO: 43 |
| 11 | INISIGYRYPPICLG | SEQ ID NO: 44 |
| 12 | IGYRYPPICLGRAPG | SEQ ID NO: 45 |
| | | SP2: (LP1) |
| 13 | YPPICLGRAPGCLMP | SEQ ID NO: 46 |
| 14 | CLGRAPGCLMPAVQN | SEQ ID NO: 47 |
| 15 | APGCLMPAVQNWLVE | SEQ ID NO: 48 |
| 16 | LMPAVQNWLVEVPTV | SEQ ID NO: 49 |
| 17 | VQNWLVEVPTVSPIS | SEQ ID NO: 50 |
| 18 | LVEVPTVSPISRFTY | SEQ ID NO: 51 |
| 19 | PTVSPISRFTYHMVS | SEQ ID NO: 52 |
| 20 | PISRFTYHMVSGMSL | SEQ ID NO: 53 |
| 21 | FTYHMVSGMSLRPRV | SEQ ID NO: 54 |
| 22 | MVSGMSLRPRVNYLQ | SEQ ID NO: 55 |
| 23 | MSLRPRVNYLQDFSY | SEQ ID NO: 56 |
| 24 | PRVNYLQDFSYQRSL | SEQ ID NO: 57 |
| | | SP3 (LP2) |
| 25 | YLQDFSYQRSLKFRP | SEQ ID NO: 58 |
| 26 | FSYQRSLKFRPKGKP | SEQ ID NO: 59 |
| 27 | RSLKFRPKGKPCPKE | SEQ ID NO: 60 |
| 28 | FRPKGKPCPKEIPKE | SEQ ID NO: 61 |
| 29 | GKPCPKEIPKESKNT | SEQ ID NO: 62 |
| 30 | PKEIPKESKNTEVLV | SEQ ID NO: 63 |
| 31 | PKESKNTEVLVWEEC | SEQ ID NO: 64 |
| 32 | KNTEVLVWEECVANS | SEQ ID NO: 65 |
| 33 | VLVWEECVANSAVIL | SEQ ID NO: 66 |
| 34 | EECVANSAVILQNNE | SEQ ID NO: 67 |
| 35 | ANSAVILQNNEFGTI | SEQ ID NO: 68 |
| 36 | VILQNNEFGTIIDWA | SEQ ID NO: 69 |
| | | SP4 (LP2) |
| 37 | NNEFGTIIDWAPRGQ | SEQ ID NO: 70 |
| 38 | GTIIDWAPRGQFYHN | SEQ ID NO: 71 |
| 39 | DWAPRGQFYHNCSGQ | SEQ ID NO: 72 |
| 40 | RGQFYHNCSGQTQSC | SEQ ID NO: 73 |
| 41 | YHNCSGQTQSCPSAQ | SEQ ID NO: 74 |
| 42 | SGQTQSCPSAQVSPA | SEQ ID NO: 75 |
| 43 | QSCPSAQVSPAVDSD | SEQ ID NO: 76 |
| 44 | SAQVSPAVDSDLTES | SEQ ID NO: 77 |
| 45 | SPAVDSDLTESLDKH | SEQ ID NO: 78 |
| 46 | DSDLTESLDKHKHKK | SEQ ID NO: 79 |
| 47 | TESLDKHKHKKLQSF | SEQ ID NO: 80 |
| 48 | DKHKHKKLQSFYPWE | SEQ ID NO: 81 |
| | | SP5 (LP3) |
| 49 | HKKLQSFYPWEWGEK | SEQ ID NO: 82 |
| 50 | QSFYPWEWGEKRIST | SEQ ID NO: 83 |
| 51 | PWEWGEKRISTPRPK | SEQ ID NO: 84 |
| 52 | GEKRISTPRPKIVSP | SEQ ID NO: 85 |
| 53 | ISTPRPKIVSPVSGP | SEQ ID NO: 86 |
| 54 | RPKIVSPVSGPEHPE | SEQ ID NO: 87 |
| 55 | VSPVSGPEHPELWRL | SEQ ID NO: 88 |
| 56 | SGPEHPELWRLTVAS | SEQ ID NO: 89 |
| 57 | HPELWRLTVASHHIR | SEQ ID NO: 90 |
| 58 | WRLTVASHHIRIWSG | SEQ ID NO: 91 |
| 59 | VASHHIRIWSGNQTL | SEQ ID NO: 92 |
| 60 | HIRIWSGNQTLETRD | SEQ ID NO: 93 |
| | | SP6 (LP3) |
| 61 | WSGNQTLETRDCKPF | SEQ ID NO: 94 |
| 62 | QTLETRDCKPFYTID | SEQ ID NO: 95 |
| 63 | TRDCKPFYTIDLNSS | SEQ ID NO: 96 |
| 64 | KPFYTIDLNSSLTVP | SEQ ID NO: 97 |
| 65 | TIDLNSSLTVPLQSC | SEQ ID NO: 98 |
| 66 | NSSLTVPLQSCVKPP | SEQ ID NO: 99 |
| 67 | TVPLQSCVKPPYMLV | SEQ ID NO: 100 |
| 68 | QSCVKPPYMLVVGNI | SEQ ID NO: 101 |
| 69 | KPPYMLVVGNIVIKP | SEQ ID NO: 102 |
| 70 | MLVVGNIVIKPDSQT | SEQ ID NO: 103 |
| 71 | GNIVIKPDSQTITCE | SEQ ID NO: 104 |
| 72 | IKPDSQTITCENCRL | SEQ ID NO: 105 |
| | | SP7 (LP4) |
| 73 | SQTITCENCRLLSCI | SEQ ID NO: 106 |
| 74 | TCENCRLLSCIDSTF | SEQ ID NO: 107 |
| 75 | CRLLSCIDSTFNWQH | SEQ ID NO: 108 |
| 76 | SCIDSTFNWQHRILL | SEQ ID NO: 109 |
| 77 | STFNWQHRILLVRAR | SEQ ID NO: 110 |
| 78 | WQHRILLVRAREGVW | SEQ ID NO: 111 |
| 79 | ILLVRAREGVWIPVS | SEQ ID NO: 112 |
| 80 | RAREGVWIPVSMDRP | SEQ ID NO: 113 |
| 81 | GVWIPVSMDRPWEAS | SEQ ID NO: 114 |
| 82 | PVSMDRPWEASPSVH | SEQ ID NO: 115 |
| 83 | DRPWEASPSVHILTE | SEQ ID NO: 116 |
| 84 | EASPSVHILTEVLKG | SEQ ID NO: 117 |
| | | SP8 (LP4) |
| 85 | SVHILTEVLKGVLNR | SEQ ID NO: 118 |
| 86 | LTEVLKGVLNRSKRF | SEQ ID NO: 119 |
| 87 | LKGVLNRSKRFIFTL | SEQ ID NO: 120 |
| 88 | LNRSKRFIFTLIAVI | SEQ ID NO: 121 |
| 89 | KRFIFTLIAVIMGLI | SEQ ID NO: 122 |
| 90 | FTLIAVIMGLIAVTA | SEQ ID NO: 123 |
| 91 | AVIMGLIAVTATAAV | SEQ ID NO: 124 |
| 92 | GLIAVTATAAVAGVA | SEQ ID NO: 125 |
| 93 | VTATAAVAGVALHSS | SEQ ID NO: 126 |
| 94 | AAVAGVALHSSVQSV | SEQ ID NO: 127 |
| 85 | GVALHSSVQSVNFVN | SEQ ID NO: 128 |
| 96 | HSSVQSVNFVNDWQK | SEQ ID NO: 129 |
| | | SP9 ((LP5) |
| 97 | QSVNFVNDWQKNSTR | SEQ ID NO: 130 |
| 98 | FVNDWQKNSTRLWNS | SEQ ID NO: 131 |
| 99 | WQKNSTRLWNSQSSI | SEQ ID NO: 132 |
| 100 | STRLWNSQSSIDQKL | SEQ ID NO: 133 |
| 101 | WNSQSSIDQKLANQI | SEQ ID NO: 134 |
| 102 | SSIDQKLANQINDLR | SEQ ID NO: 135 |
| 103 | QKLANQINDLRQTVI | SEQ ID NO: 136 |
| 104 | NQINDLRQTVIWMGD | SEQ ID NO: 137 |
| 105 | DLRQTVIWMGDRLMS | SEQ ID NO: 138 |
| 106 | TVIWMGDRLMSLEHR | SEQ ID NO: 139 |
| 107 | MGDRLMSLEHRFQLQ | SEQ ID NO: 140 |
| 108 | LMSLEHRFQLQCDWN | SEQ ID NO: 141 |
| | | SP10 (LP5) |
| 109 | EHRFQLQCDWNTSDF | SEQ ID NO: 142 |
| 110 | QLQCDWNTSDFCITP | SEQ ID NO: 143 |
| 111 | DWNTSDFCITPQIYN | SEQ ID NO: 144 |
| 112 | SDFCITPQIYNESEH | SEQ ID NO: 145 |
| 113 | ITPQIYNESEHHWDM | SEQ ID NO: 146 |
| 114 | IYNESEHHWDMVRRH | SEQ ID NO: 147 |
| 115 | SEHHWDMVRRHLQGR | SEQ ID NO: 148 |
| 116 | WDMVRRHLQGREDNL | SEQ ID NO: 149 |
| 117 | RRHLQGREDNLTLDI | SEQ ID NO: 150 |
| 118 | QGREDNLTLDISKLK | SEQ ID NO: 151 |
| 119 | DNLTLDISKLKEQIF | SEQ ID NO: 152 |
| 120 | LDISKLKEQIFEASK | SEQ ID NO: 153 |
| | | SP11 (LP6) |
| 121 | KLKEQIFEASKAHLN | SEQ ID NO: 154 |
| 122 | QIFEASKAHLNLVPG | SEQ ID NO: 155 |
| 123 | ASKAHLNLVPGTEAI | SEQ ID NO: 156 |
| 124 | HLNLVPGTEAIAGVA | SEQ ID NO: 157 |
| 125 | VPGTEAIAGVADGLA | SEQ ID NO: 158 |
| 126 | EAIAGVADGLANLNP | SEQ ID NO: 159 |
| 127 | GVADGLANLNPVTWV | SEQ ID NO: 160 |
| 128 | GLANLNPVTWVKTIG | SEQ ID NO: 161 |
| 129 | LNPVTWVKTIGSTTI | SEQ ID NO: 162 |
| 130 | TWVKTIGSTTIINLI | SEQ ID NO: 163 |
| 131 | TIGSTTIINLILILV | SEQ ID NO: 164 |
| 132 | TTIINLILILVCLFC | SEQ ID NO: 165 |
| | | SP12 (LP6) |
| 133 | NLILILVCLFCLLLV | SEQ ID NO: 166 |
| 134 | ILVCLFCLLLVCRCT | SEQ ID NO: 167 |
| 135 | LFCLLLVCRCTQQLR | SEQ ID NO: 168 |
| 136 | LLVCRCTQQLRRDSD | SEQ ID NO: 169 |
| 137 | RCTQQLRRDSDHRER | SEQ ID NO: 170 |
| 138 | QLRRDSDHRERAMMT | SEQ ID NO: 171 |
| 139 | DSDHRERAMMTMAVL | SEQ ID NO: 172 |
| 140 | RERAMMTMAVLSKRK | SEQ ID NO: 173 |
| 141 | MMTMAVLSKRKGGNV | SEQ ID NO: 174 |
| 142 | AVLSKRKGGNVGKSK | SEQ ID NO: 175 |
| 143 | KRKGGNVGKSKRDQI | SEQ ID NO: 176 |
| 144 | GNVGKSKRDQIVTVSV | SEQ ID NO: 177 |

As used herein, an “amino acid molecule” or “amino acid residue” refers to any naturally occurring amino acid, any amino acid derivative or any amino acid mimic known in the art, including modified or unusual amino acids. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties. In specific aspects, the composition of the present invention employs a peptide of from about 5 to about 100 amino acids or greater in length.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

The term “peptide” is used interchangeably with “oligopeptide” in the present specification to designate a series of residues, typically L-amino acids, connected one to the other, typically by peptide bonds between the α-amino and carboxyl groups of adjacent amino acids. The oligopeptides of the invention may be about 15 residues or less in length and may consist of between about 9 and about 11 residues, including 9, 10 and 11 residues. The oligopeptides may be less than about 50 residues in length and range from between about 9 to about 30 residues, between about 15 and 25 residues, and between about 18 and 20 residues.

In certain embodiments the size of the at least one peptide molecule may comprise, but is not limited to, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, or greater amino molecule residues, and any range derivable therein.

An “immunogenic peptide” or “peptide epitope” is a peptide which comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

Accordingly, the term “proteinaceous composition” encompasses amino molecule sequences comprising at least one of the 20 common amino acids in naturally synthesized proteins, or at least one modified or unusual amino acid, including but not limited to those shown on Table 3 below.

TABLE 3
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | 2-alanine, -Amino- | Ahyl | Allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | Allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

A. Fusion Proteins of Peptides

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

B. Variants of Peptides

It is contemplated that peptides of the present invention may further employ amino acid sequence variants such as substitutional, insertional or deletion variants. Deletion variants lack one or more residues of the native protein. Insertional mutants typically involve the addition of material at a non-terminal point in the polypeptide. Substitutions are changes to an existing amino acid. These sequence variants may generate truncations, point mutations, and frameshift mutations. As is known to one skilled in the art, synthetic peptides can be generated by these mutations.

It also will be understood that amino acids sequence variants may include additional residues, such as additional N- or C-terminal amino acids, and yet still be essentially as set forth in one of the sequences disclosed herein, so long as the sequence meets the criteria set forth above, including the maintenance of biological activity.

The following is a discussion based upon changing the amino acids of a protein, such as a peptide or protein of the invention, to create a mutated, truncated, or modified protein. For example, certain amino acids may be substituted for other amino acids in the tumor-associated peptide or protein, resulting in a greater CTL immune response in cells such as a myeloid cell. Since it is the interactive capacity and nature of a protein that defines that protein's biological functional activity, certain amino acid substitutions can be made in a protein sequence, and in its underlying nucleic acid coding sequence, thereby producing a mutated, truncated or modified protein.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention may also employ the use of peptide mimetics for the preparation of polypeptides (see e.g., Johnson, 1993) having many of the natural properties of a tumor-associated HLA-restricted peptide. The underlying rationale behind the use of peptide mimetics is that the peptide backbone of proteins exists chiefly to orient amino acid side chains in such a way as to facilitate molecular interactions, such as those of antibody and antigen. These principles may be used, in conjunction with the principles outline above, to engineer second generation molecules having many of the natural properties of a tumor-associated peptide or polypeptide but with altered and even improved characteristics.

C. Tumor-Associated Peptide Purification

In certain embodiments the protein(s) of the present invention may be purified. It may be desirable to purify the tumor-associated peptides, polypeptides or proteins or variants thereof. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying a tumor-associated peptide or polypeptide of the present invention, it may be desirable to express the polypeptide in a prokaryotic or eukaryotic expression system and extract the protein using denaturing conditions. The polypeptide may be purified from other cellular components using an affinity column, which binds to a tagged portion of the polypeptide. Although this preparation will be purified in an inactive form, the denatured material will still be capable of transducing cells. Once inside of the target cell or tissue, it is generally accepted that the polypeptide will regain full biological activity.

As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### IV. VACCINE COMPONENTS

In other embodiments of the invention, the antigenic composition, such a tumor-associated polypeptide, peptide or antigen, may comprises an additional immunostimulatory agent or nucleic acids encoding such an agent Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. Other immunopotentiating compounds are also contemplated for use with the compositions of the invention such as polysaccharides, including chitosan, which is described in U.S. Pat. No. 5,980,912, hereby incorporated by reference. Multiple (more than one) tumor-associated epitopes may be crosslinked to one another (e.g., polymerized). The use of small peptides for antibody generation or vaccination also typically requires conjugation of the peptide to an immunogenic carrier protein.

One of ordinary skill would know various assays to determine whether an immune response against a tumor-associated peptide was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al. (1991), hereby incorporated by reference.

A. Adjuvants

As also well known in the art, the immunogenicity of a particular immunogen composition can be enhanced by the use of non-specific stimulators of the immune response, known as adjuvants. Adjuvants have been used experimentally to promote a generalized increase in immunity against unknown antigens (e.g., U.S. Pat. No. 4,877,611) Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. For many cancers, there is compelling evidence that the immune system participates in host defense against the tumor cells, but only a fraction of the likely total number of tumor-specific antigens are believed to have been identified to date. The use of the tumor-associated antigens of the present invention with the inclusion of a suitable adjuvant will likely increase the anti-tumor response of the antigens. Suitable molecule adjuvants include all acceptable immunostimulatory compounds, such as cytokines, toxins or synthetic compositions.

Exemplary, often preferred adjuvants include complete Freund's adjuvant (a non-specific stimulator of the immune response containing killed Mycobacterium tuberculosis), incomplete Freund's adjuvants and aluminum hydroxide adjuvant. Other adjuvants that may also be used include IL-1, IL-2, IL-4, IL-7, IL-12, interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopol®) used as an about 0.25% solution. Adjuvant effect may also be made my aggregation of the antigen in the vaccine by heat treatment with temperatures ranging between about 70° to about 101° C. for a 30 second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

Some adjuvants, for example, certain organic molecules obtained from bacteria, act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. It is thought to be effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present invention.

BCG (bacillus Calmette-Guerin, an attenuated strain of Mycobacterium) and BCG-cell wall skeleton (CWS) may also be used as adjuvants, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945. BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martin et al., 1990). Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface active agents, e.g., saponin and derivatives such as QS21 (Cambridge Biotech), form yet another group of adjuvants for use with the immunogens of the present invention. Nonionic block copolymer surfactants (Rabinovich et al., 1994) may also be employed. Oligonucleotides are another useful group of adjuvants (Yamamoto et al., 1988). Quil A and lentinen are other adjuvants that may be used in certain embodiments of the present invention.

Another group of adjuvants are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the invention (Takada et al., 1995).

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

Adjuvants may be encoded by a nucleic acid (e.g., DNA or RNA). It is contemplated that such adjuvants may be also be encoded in a nucleic acid (e.g., an expression vector) encoding the antigen, or in a separate vector or other construct. Nucleic acids encoding the adjuvants can be delivered directly, such as for example with lipids or liposomes.

B. Biological Response Modifiers

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m2) (Johnson/Mead, NJ), cytokines such as -interferon, IL-2, or IL-12 or genes encoding proteins involved in immune helper functions, such as B-7.

C. Chemokines

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemoattractant effects and could also be classified under the term chemokines.

D. Immunogenic Carrier Proteins

In certain embodiments, an antigenic composition may be chemically coupled to a carrier or recombinantly expressed with a immunogenic carrier peptide or polypetide (e.g., a antigen-carrier fusion peptide or polypeptide) to enhance an immune reaction. Exemplary and preferred immunogenic carrier amino acid sequences include hepatitis B surface antigen, keyhole limpet hemocyanin (KLH) and bovine serum albumin (BSA). Other albumins such as ovalbumin, mouse serum albumin or rabbit serum albumin also can be used as immunogenic carrier proteins. Means for conjugating a polypeptide or peptide to a immunogenic carrier protein are well known in the art and include, for example, glutaraldehyde, m-maleimidobenzoyl-N-hydroxysuccinimide ester, carbodiimide and bis-biazotized benzidine.

### V. NUCLEIC ACIDS ENCODING PEPTIDES AND POLYPEPTIDES

It is contemplated in the present invention, that the tumor-associated peptides, or polypeptides may be encoded by a nucleic acid sequence. A nucleic acid may be derived from genomic DNA, complementary DNA (cDNA) or synthetic DNA. Where incorporation into an expression vector is desired, the nucleic acid may also comprise a natural intron or an intron derived from another gene.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy. A tumor-associated peptide or polypeptide cDNA, for use in the present invention, may be derived from human cDNA but are not limited such.

As used herein, the term “nucleic acid segment” refers to a nucleic acid molecule that has been isolated free of total genomic DNA of a particular species. Therefore, a nucleic acid segment encoding a polypeptide refers to a nucleic acid segment that contains wild-type, polymorphic, or mutant polypeptide-coding sequences yet is isolated away from, or purified free from, total mammalian or human genomic DNA. Included within the term “nucleic acid segment” are a polypeptide or polypeptides, DNA segments smaller than a polypeptide, and recombinant vectors, such as, plasmids and other non-viral vectors. The term “recombinant” may be used in conjunction with a polypeptide or the name of a specific polypeptide, and generally refers to a polypeptide produced from a nucleic acid molecule that has been manipulated in vitro or that is the replicated product of such a molecule.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length. Those of skill will recognize that in cases where the nucleic acid region encodes a tumor-associated HLA-restricted peptide, or polypeptide, the nucleic acid region can be quite long, depending upon the number of amino acids in the fusion protein.

It is contemplated that the tumor-associated peptide, or polypeptide may be encoded by any nucleic acid sequence that encodes the appropriate amino acid sequence. The design and production of nucleic acids encoding a desired amino acid sequence is well known to those of skill in the art, using standardized codon tables (Table 3). In preferred embodiments, the codons selected for encoding each amino acid may be modified to optimize expression of the nucleic acid in the host cell of interest. The term “functionally equivalent codon” is used herein to refer to codons that encode the same amino acid, such as the six codons for arginine or serine, and also refers to codons that encode biologically equivalent amino acids. Codon preferences for various species of host cell are well known in the art. Codons preferred for use in humans, are well known to those of skill in the art (Wada et. al., 1990). Codon preferences for other organisms also are well known to those of skill in the art (Wada et al., 1990, included herein in its entirety by reference).

TABLE 4
Codon Table

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Prokaryote- and/or eukaryote-based systems can be used to produce nucleic acid sequences, or their cognate polypeptides, proteins and peptides. The present invention contemplates the use of such an expression system to produce the tumor-associated HLA-restricted peptide, or polypeptide. More specifically, the present invention employs the use of the insect cell/baculovirus system. The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the expression system disclosed in the invention, numerous expression systems exists which are commercially and widely available. One example of such a system is the STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

A. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986).

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the vaccines of the present invention (Muzyczka, 1992). AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as gene delivery vectors in vaccines due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding an antigen of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Nucleic Acid Delivery

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### VI. PHARMACEUTICAL VACCINE FORMULATION AND DELIVERY

In an embodiment of the present invention, a method of treatment and prevention of cancers such as breast and ovarian by the delivery of a tumor-associated peptide, polypeptide or expression constructs therefore is contemplated. Examples of other cancers contemplated for treatment include lung cancer, head and neck cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, bladder cancer and any other neoplastic diseases that may be treated or prevented by a tumor-associated peptides or polypeptides of the present invention.

An effective amount of the pharmaceutical vaccine composition, generally, is defined as that amount sufficient to detectably and repeatedly to ameliorate, reduce, minimize or limit the extent of the disease or condition or symptoms thereof. More rigorous definitions may apply, including elimination, eradication or cure of disease.

Preferably, patients will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

A. Vaccine Administration

To kill cells, inhibit cell growth, inhibit metastasis, decrease tumor or tissue size and otherwise reverse or reduce the malignant phenotype of tumor cells, using the methods and compositions of the present invention, one would generally contact a cancer cell with the therapeutic compound such as a polypeptide or an expression construct encoding a polypeptide. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation. Any of the formulations and routes of administration discussed with respect to the treatment or diagnosis of cancer may also be employed with respect to neoplastic diseases and conditions.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a tumor-associated peptide, polypeptide or construct encoding therefor. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hr, to about 2-6 hr, to about 6-12 hr, to about 12-24 hr, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

B. Injectable Compositions and Formulations

One method for the delivery of a pharmaceutical according to the present invention is systemically. However, the pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Injection of pharmaceuticals may be by syringe or any other method used for injection of a solution, as long as the agent can pass through the particular gauge of needle required for injection. A novel needleless injection system has been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermolysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### VII. COMBINATION TREATMENTS

The compounds and methods of the present invention may be used in the context of neoplastic diseases/conditions including cancer. Types of cancers may include lung cancer, head and neck cancer, breast cancer, pancreatic cancer, prostate cancer, renal cancer, liver cancer, bone cancer, ovarian cancer, testicular cancer, cervical cancer, gastrointestinal cancer, lymphomas, pre-neoplastic lesions in the lung, colon cancer, melanoma, bladder cancer and any other neoplastic diseases. In order to increase the effectiveness of a treatment with the tumor-associated antigen compositions of the present invention, it may be desirable to combine these compositions with other agents effective in the treatment of those diseases and conditions. For example, the treatment of a cancer may be implemented with therapeutic compounds of the present invention and other anti-cancer therapies, such as anti-cancer agents or surgery.

Various combinations may be employed; for example, the tumor-associated antigen is “A” and the secondary anti-cancer is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic agents of the present invention to a patient will follow general protocols for the administration of that particular secondary therapy, taking into account the toxicity, if any, of the tumor-associated antigen treatment. It is expected that the treatment cycles would be repeated as necessary. It also is contemplated that various standard therapies, as well as surgical intervention, may be applied in combination with the described cancer cell.

A. Adjunct Anti-Cancer Therapy

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that tumor-associated HLA-restricted peptide therapy could be used similarly in conjunction with chemotherapeutic, radiotherapeutic, immunotherapeutic or other biological intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of Fortilin would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons, and; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the tumor-associated HLA-restricted peptide therapies described herein.

i) Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

ii) Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

4. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Epitope Mapping.

The inventor used epitope mapping to identify responses to HERV-K peptides in breast cancer patients. They chemically synthesized a HERV-K env nine amino acid (9mer) peptide library (33 HLA A2 restricted peptides) and overlapping 15mer peptide library (144 peptides). Preliminary research has identified two pools from the HERV-K env 9mer peptide library that contain potential CD8+ epitopes, using interferon gamma (IFN-γ) enzyme-linked immunosorbent spot (ELISPOT) assay with T cells from one HLA A2⁺ normal donor which was stimulated with the 9mer peptide pool-pulsed homologous dendritic cells (DCs).

Screening of HERV-K specific T and B cell specific epitopes will be a major goal. The approach is to initially identify ovarian cancer patients as a source of samples. The inventor will further identify candidate epitopes from the above two positive pools in breast or ovarian cancer patients. The findings of both specific cellular and humoral responses to 9mer/15mer peptide pools in breast cancer patients have led us to hypothesize that 9mer and 15mer peptide libraries may contain HERV-K specific T and B cell epitopes. These data provide strong evidence that HERV-K specific T and B cell specific epitopes can be identified by IFNγ ELISPOT and enzyme-linked immunosorbent assay (ELISA) using in vitro stimulated (IVS) cells and sera obtained from breast cancer patients. Active peptides will be subjected to the immune response studies described below.

Determining Sensitivity of Ovarian Cancer Cells to Cd4⁺, Cd25⁺ T-Regulatory Cell Suppression and Develop Tumor-Infiltrating Lymphocyte Adoptive Therapy for Ovarian Cancer.

TILs isolated from tumor tissue and cultured with interleukin-2 have been demonstrated to exhibit re-infiltration of the tumor, and they may induce lysis of tumor cells and tumor regression. Adoptive cell therapy using TIL for metastatic melanoma has shown objective response rates as high as 72%. The use of therapeutic TILs has been considered for adoptive immunotherapy. CD4⁺CD25⁺Foxp3⁺ regulatory T (Treg) cells have been shown to play important roles in mediating cancer development. Under pathologic conditions, cancers can use Treg cells for immune evasion. The number of functional Treg cells is elevated in cancer patients, leading to the observed immunosuppression. Therefore, to potentiate elimination of tumors by the immune system, targeting of Treg cells may be beneficial. TIL derived from human tumors have been successfully cultured from breast cancer surgical biopsies in the inventor's laboratory. These TIL cells derived from human tumors were further characterized by flow cytometry. For example, CD4 (N/T: 82.47%/82.17%), CD8 (N/T:0%/48.04%), CD25 (N/T: 5.67%/5.47%), FoxP3 (N/T: 88.7%/96.11%) and CD56 (N/T: 0%/43.68%) were detected in TIL cells obtained from breast cancer (T) and matched uninvolved tissues (N). Higher percentages of CD8, CD56 and FoxP3 TIL cells were detected in tumor tissues than in matched uninvolved tissues. These data indicate that these TIL cells cultured from breast cancer with elevated CD8 T and NK cells can be used directly in breast cancer patients for adoptive therapy against their own tumors, and these results will be verified in ovarian cancer patients.

Studies Determining CD8⁺ T Cell Reactivity.

Cancer patients were enrolled to evaluate HERV-K as a tumor associated antigen target. Patients provided written informed consent as part of an approved protocol (LAB04-0083) at MD Anderson Cancer Center prior to commencing the studies. Normal female healthy donor control blood samples were obtained from Gulf Coast Regional Blood Center, Houston Tex. 40 mL of peripheral blood was obtained from each patient, and peripheral blood mononuclear cells (PBMCs) were isolated by density gradient centrifugation using Histopaque-1077 (Sigma-Aldrich; USA). HLA-A*02 expression in PBMCs obtained from cancer patients and healthy subjects was determined by flow cytometry with PE Mouse anti-human HLA-A2 mAb BB7.2 (BD Pharmingen; San Diego, Calif., USA). Mouse IgG2b was used as an isotype control. PBMCs (5×10⁵ cells/well) from either healthy subjects or cancer patients were incubated with Peptide #18-135 (IINLILILVCLFCLLLVCRCTQQLR; SEQ ID NO: 178), Peptide #73-76 (SQTITCENCRLLSCIDSTFNWQHRILL; SEQ ID NO: 179) or KLH as control peptide) or proteins (HERV-K env SU protein or GST protein as control) at concentrations of 40 μg/mL in 96-well U-bottom microplates (BD; Franklin Lakes, N.J., USA) in 100 μL of T-cell medium (TCM), consisting of RPMI 1640 (Mediatech; Manassas, Va., USA), 10% human AB serum (Valley Biomedical, Winchester, USA), 50 mM 2-mercaptoethanol, 100 IU/mL interleukin-2 (IL-2), and 0.1 mM MEM nonessential amino acid solution (Invitrogen; Grand Island, N.Y., USA). Half of the TCM was removed and replaced with fresh TCM containing peptides (20 μg/mL) on day 7. After 14 days of culture, the supernatants from cell culture media (PBMC pulsed with peptides or proteins) were harvested and tested for their ability to produce IFN-gamma or IL-2, and granzyme B release was also determined by enzyme-linked immunosorbent assay (ELISA) assay. In order to detect the secreted cytokine in this assay, high-binding plates were coated with 5 μg/ml anti-IFN-γ (Mabtech, Stockholm, Sweden) diluted in PBS buffer at 4° C. overnight. After three washes with PBS, the wells were blocked with 100 μL per well of 3% bovine serum albumin (BSA) for 120 min in room temperature. Then 50 μL supernatant were added per well. After 20 h of incubation at 4° C., supernatants were removed by flicking the plate, followed by repeated washings with PBS. To detect captured IFN-γ, the inventor added 1 μg/ml biotinylated detection MAb (Mabtech) diluted in PBS containing 0.5% Tween 20 and 3% BSA per well. After 1 h of incubation at 37° C. and 6 washes, 100 μl of avidin-biotin alkaline phosphatase complex (ABC-AP; Vector Laboratories, Burlingame, Calif.) diluted 1:1000 in PBS was added for 30 min. Unbound ABC-AP was removed by 6 consecutive washes with PBS, and 100 μl of freshly prepared enzyme substrate solution PNPP was added to each well. Absorbance was measured after 10 min of incubation in the dark and the plate was read on a Wallac Victor 2 V Microplate Reader (PerkinElmer, Waltham, Mass.). The peptides were synthesized by a solid-phase method by Peptide 2 Inc with purification >95%. The synthesized peptides were dissolved in DMSO at a concentration of 10 mg/mL and stored at −80° C. until further use.

HERV-K peptide-specific T cells were expanded by a rapid expansion protocol (REP). Briefly, on day 0, 0.1×10⁶-0.5×10⁶ HERV-K peptide specific T cells were cultured in a T25 flask with 20 mL RPMI-1640 supplemented with 10% human AB serum, 50 mM of 2-mercaptoethanol, and 30 ng/mL OKT3 antibody (Abcam Inc., Cambridge, Mass.), together with 20×10⁶ irradiated allogeneic PBMCs. Flasks were incubated upright at 37° C. in 5% CO2. IL-2 (300 IU/mL) was added on day 1, and on day 5, half of the cell culture supernatant was removed and replenished with fresh medium containing 300 IU/mL IL-2. 14 days after initiation of the REP, cells were harvested and cryopreserved for future experiments.

Human Patient and Normal Donor PBMC Isolation and Antigen Pulsing.

Heparinated human patient blood was collected at M.D. Anderson Cancer Center via IRB-approved protocols through informed consent. Heparinated normal donor blood was purchased from Gulf Coast Regional Blood Center (Houston, Tex.). Buffy coats were isolated from heparinated blood samples using histopaque 1077 (Sigma, St. Louis Mo.) as per manufacturer's instructions. The recovered PBMCs were pelleted, treated with red blood cell (RBC) lysis buffer, washed twice with PBS, and seeded into 96 well round-bottom plates at 200,000 cells per well in complete RPMI-1640 containing 10% human serum, non-essential amino acids (NEAA), pen/strep/amphotericin B, 100 U/ml rhIL-2, and 50 μM (β-mercaptoethanol overnight (T cell basal media). The next day, half of the media was removed from each well and replaced with the same basal media formulation containing 80 μg/ml HERV-K Env peptides or recombinant K10 g protein. After one week, half the media was again removed and replaced with the same T cell basal media containing 40 μg/ml of the same peptide or protein. One week later half the media was again removed and assayed for interferon gamma by sandwich ELISA.

Interferon γ ELISA.

High-binding 96 well microplates were coated with 5 μg/ml anti-human interferon gamma antibody (MabTech, Mariemont, Ohio, clone 1-D1K) capture antibody overnight. The plate was rinsed and blocked with 3% BSA in TBS-0.2% tween 20 (blocker) for 1 hour. The blocking solution was decanted and 50 μl of conditioned media from each well of the antigen pulsing plate was added and incubated overnight. The next day the plates were washed in TBS-tween 20 and 1 μg/ml biotinylated anti-human interferon gamma detection antibody (MabTech, clone 7-B6-1) was added in blocker for 2 hours and rinsed. The plates were then incubated with extravidin-alkaline phosphatase (Sigma) diluted 1:10,000 in blocker for 1 hour. The plates were rinsed again with TBS-tween 20 followed by TBS. 100 μl pNPP substrate (Sigma) was then added; the plate was read in absorbance mode at 405 nm using a Victor V plate reader (Perkin-Elmer, Waltham, Mass.) after 30 minutes and overnight incubation.

T Cell Sorting and Expansion.

The wells with the highest positive interferon gamma result (over one standard deviation above the group average) were pooled and stained with anti-human CD8-APC (BD Biosciences, San Jose, Calif.) or isotype control along with the viability stain EthD-1 (Life Technologies, Grand Island, N.Y.). CD8⁺EthD-1⁻ events were sorted using a FACSAria II (BD) directly into 96 well plates pre-coated with 1 μg/ml anti-human CD3 antibody (ATCC, Manassas, Va., clone Okt-3) containing T cell expansion media and 20,000 lethally-irradiated K562 aAPCs overexpressing both HERV-K Env and IL-15. These plates were monitored continually for T cell colony outgrowth.

### Example 2

### Results

Identification of HERV-K HLA Class I (HLA-A*0201) Epitopes Stimulating CD8+ Responses in Normal Donors and Ovarian Cancer Patients and their Sensitivity to CD4, CD25 T-Regulatory (T-Reg) Cell Suppression.

The inventor has tested for the presence of anti-HERV-K T-cell responses in human peripheral blood mononuclear cells (PBMCs) from ovarian cancer patients and normal donors. The inventor used an in vitro system to determine whether a cytotoxic T lymphocyte (CTL) immune response can be elicited in ovarian cancer patients. Dendritic cells were generated from adherent PBMCs in cultures containing the cytokine combination of GM-CSF and IL-4 Immature dendritic cells were pulsed with or without HERV-K proteins or cRNA and TNF-α for maturation. The inventor compared the level of HERV-K-specific T-cell responses in normal female donors, or benign ovarian disease, to ovarian cancer patients using in vitro stimulated (IVS) PBMC. The dendritic cells were pulsed with HERV-K cRNA and mixed with autologous PBMC for 7 days to generate singly-stimulated IVS cells, as described previously (Wang-Johanning et al., 2008). HERV-K-specific T-cell proliferation and CTL activity was determined, using INF-γ ELISPOT.

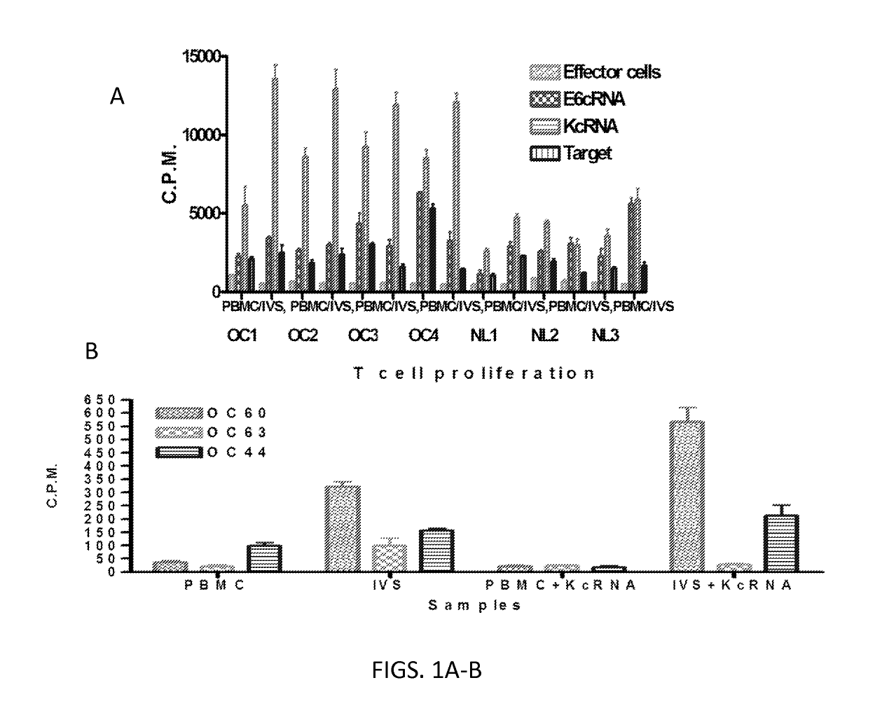

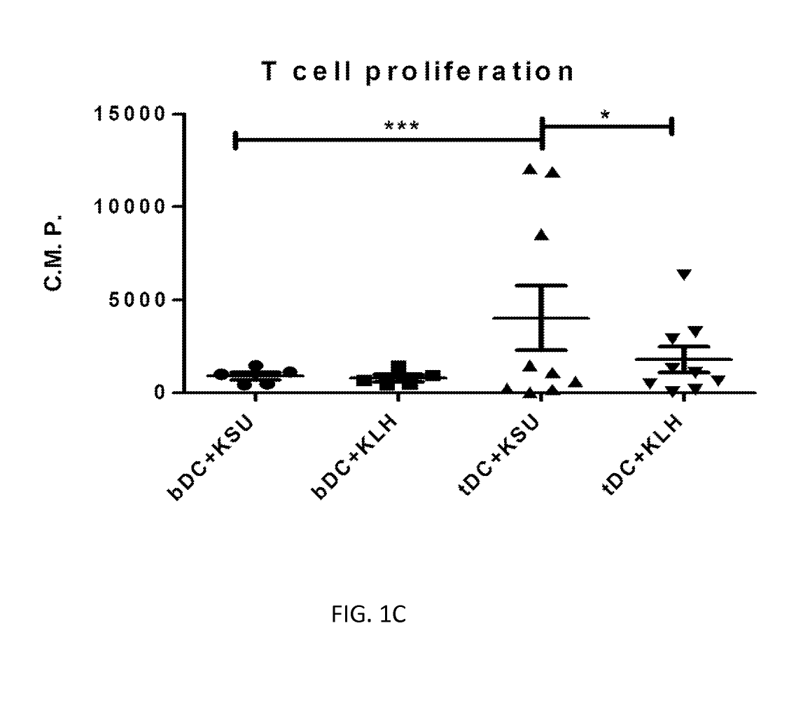

T cell proliferation was compared in freshly-isolated (ex vivo) PBMC versus IVS cells pulsed with HERV-K cRNA from 4 ovarian cancer patients and 3 normal donors. T cell proliferation specific to HERV-K env pulsed dendritic cells as antigen-presenting cells was compared between PBMC and IVS cells obtained from the same donors by a ³H-thymidine incorporation assay. Marked HERV-K-specific proliferation was detected after IVS of PBMC from ovarian cancer patients (OC 1 to OC 4, FIG. 1A), with a significant difference between dendritic cells pulsed with HERV-K env cRNA or HPV E6 cRNA (as control).

No HERV-K-specific proliferation was detected after IVS of PBMC from normal donors (NL1 to NL 3). As shown in FIG. 1A, proliferation was higher in IVS obtained from ovarian cancer patients than in IVS obtained from control subjects, which indicates that HERV-K env protein is able to induce CD4⁺ T cell response in ovarian cancer patients. Another example is shown in FIG. 1B. Higher HERV-K-specific T cell responses were found in PBMC and IVS cells obtained from ovarian cancer patients (OC 60 and OC 44) than from a patient with teratoma (OC 63). This indicates that ovarian cancer patients had a strong T cell response to HERV-K antigen. A summary of results is shown in FIG. 1C.

As shown in FIGS. 1A-C, proliferation was higher in IVS obtained from ovarian cancer patients than in IVS obtained from control subjects, including normal female donors or benign patients, indicating that HERV-K env protein is able to induce a CD4⁺ T cell response in ovarian cancer patients.

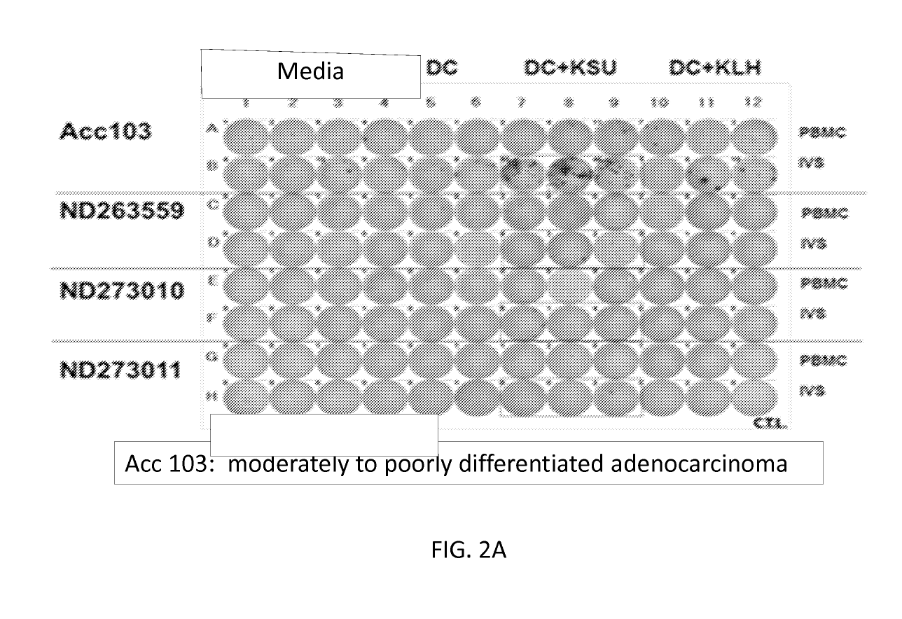

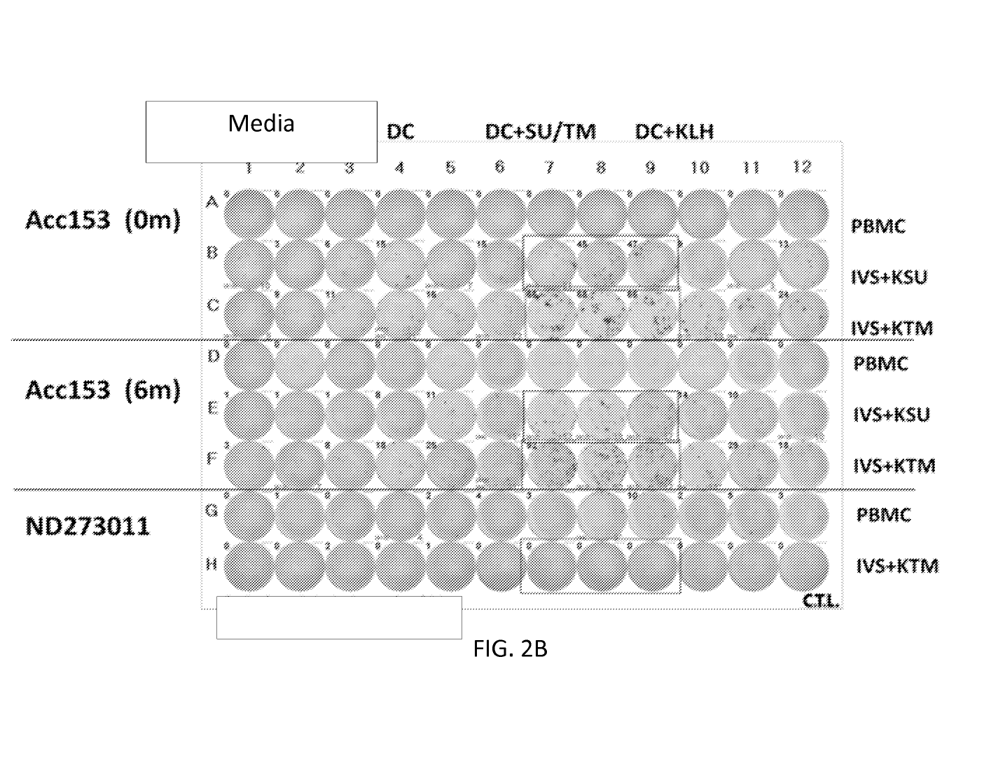

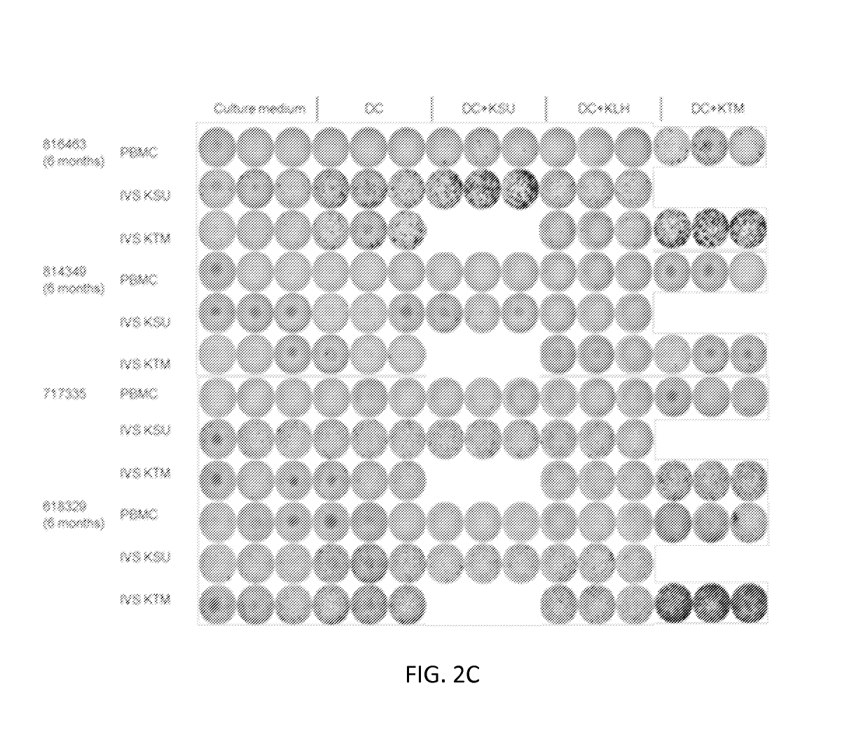

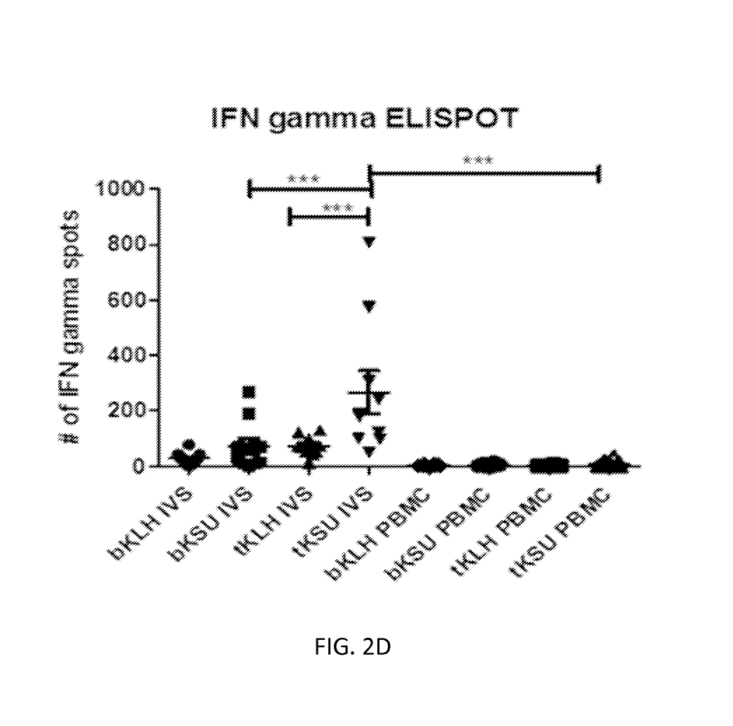

IFN-γ ELISPOT was used to determine HERV-K-specific T cell response. IFN-γ spots were detected in IVS cells (1×10⁵ cells loaded) from ovarian cancer patients but not in normal female donors stimulated one time with a single HERV-K env antigen. Three IFN-γ ELISPOTs are shown in FIGS. 2A-D. Higher spot numbers were found in patient Acc 103, who has moderately to poorly differentiated adenocarcinoma, compared with three normal female donors (ND263559, 273010, and 273011). A higher number of spots for HERV-K surface protein (KSU) (red box) or HERV-K transmembrane protein (KTM) (green box) were found in patient Acc 153, who has high grade papillary serous carcinoma, before surgery (0 m) or 6 months after surgery (6 m), in comparison to the number of spots for one normal female donor (ND 273011) (FIG. 2B). This result indicates that the T cell response to HERV-K antigen persists for at least 6 months in ovarian cancer patients. Significant differences were found between PBMC and their IVS obtained from ovarian cancer patients (P=0.0042, N=7; FIG. 2C), but no significant differences were observed in PBMC and IVS from normal female donors (N=8). There was also a significant increase in IFN-γ spots in IVS PBMC obtained from ovarian cancer patients in comparison to normal female donors (P=0.0067, data not shown) and to female donors with benign diseases (FIG. 2D). These data indicate that HERV-K antigen is a tumor specific antigen for ovarian cancer. The inventor next determined the ability of ovarian cancer patient PBMC to produce additional cytokines in response to re-stimulation with HERV-K in vitro.

Significantly more spots were found in PBMC than in autologous IVS cells obtained from ovarian cancer patients, but no significant differences in spot numbers were observed between PBMC and IVS cells from female normal donors or donors with benign diseases. In addition, significantly more spots were detected in IVS cells from ovarian cancer patients (tKSU IVS) than patients with benign disease (bKSU IVS). IVS cells were generated from PBMC stimulated with a single dose of HERV-K SU antigen.

CTL Assay.

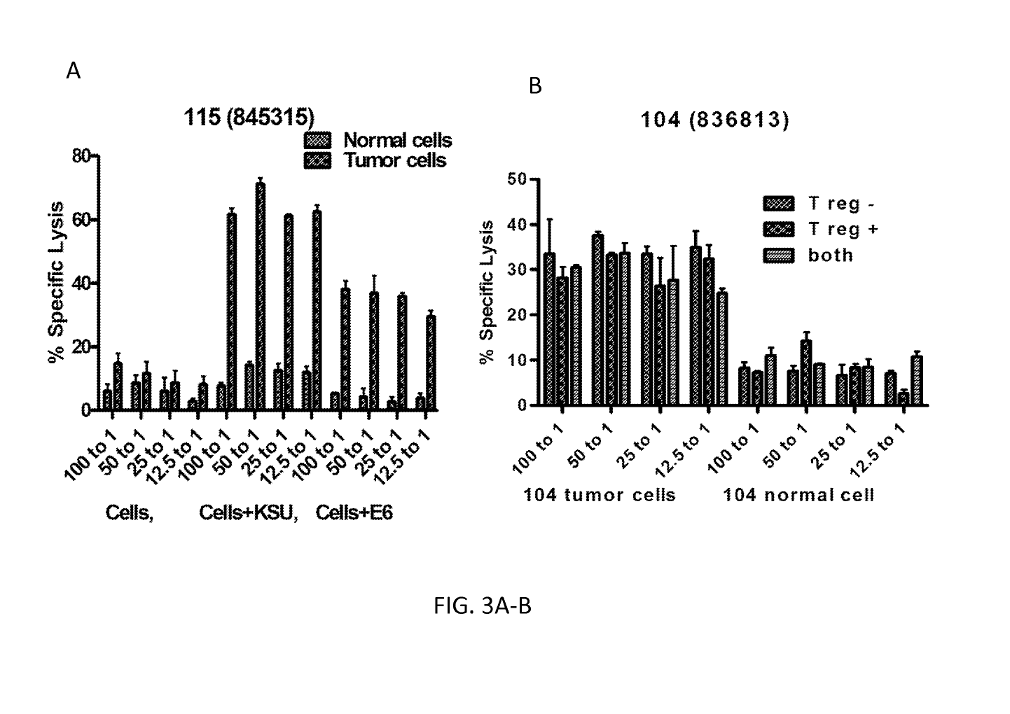

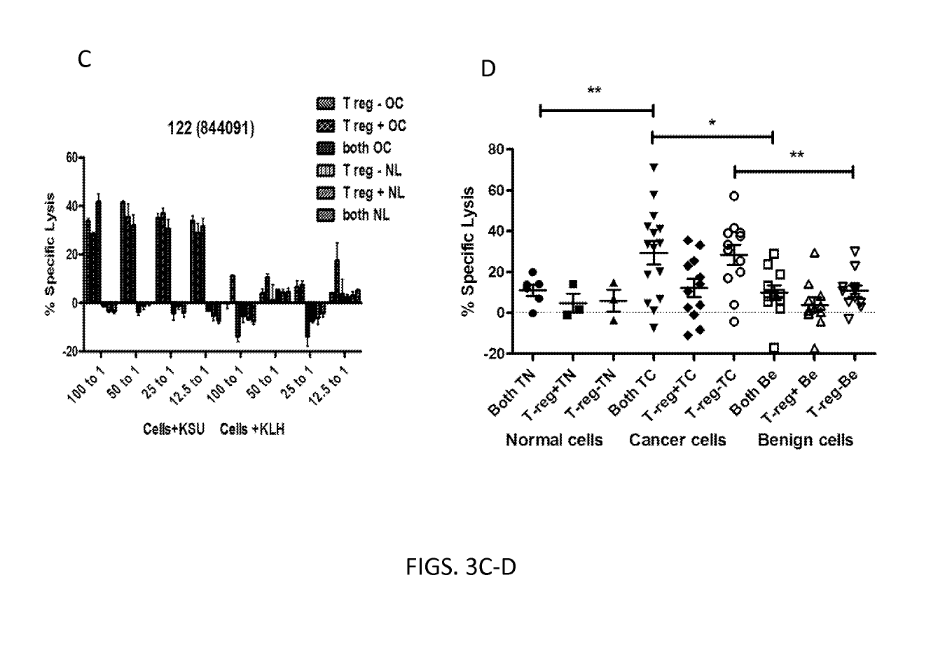

The inventor next determined the ability of HERV-K specific T cells generated from ovarian cancer patient PBMCs to lyse their own tumor cells, but not their own normal cells. Since the inventor was successful in attempts to culture primary cancer or normal epithelial cells from ovarian tumor biopsies or matched uninvolved ovarian tissues, these cells can now be used as target cells, and examine the antitumor effects of HERV-K specific T cells. Three examples of this approach are shown in FIGS. 3A-D.

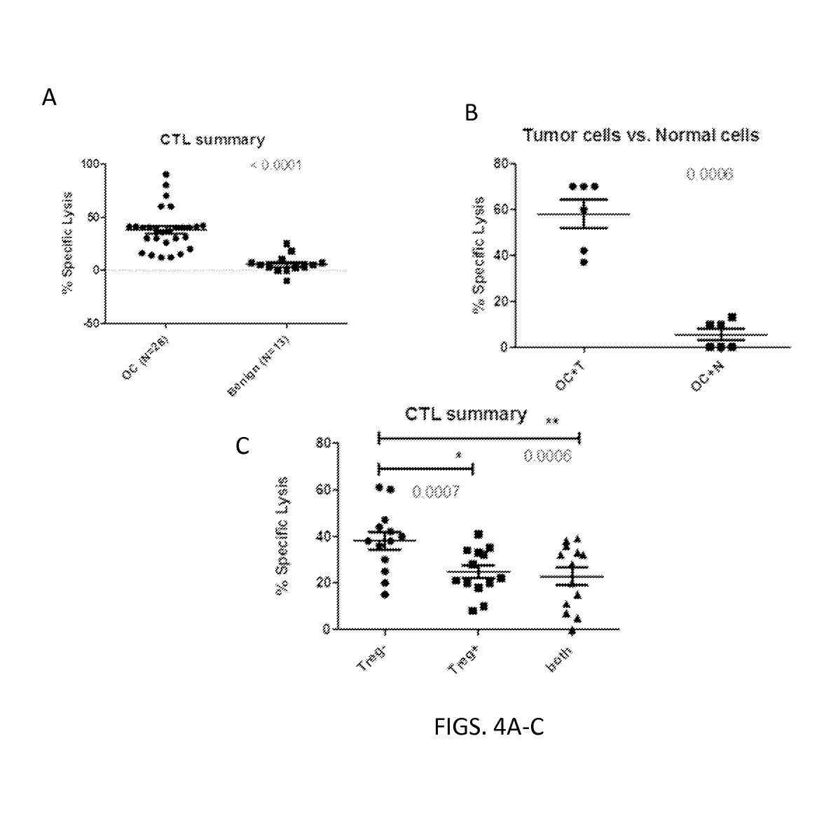

More than 70% of IVS cells were able to lyse their own tumor cells (20 to 70% specific lysis, N=15). No cytotoxicity was detected in their matched non-malignant cells (N=6), and IVS cells obtained from benign ovarian disease did not exhibit cytotoxicity toward their own primary cells (N=12). In addition, 50% of IVS PBMC showed enhanced cytotoxicity toward their own tumor cells after regulatory T cells (Treg) were depleted (N=12). FIG. 3D indicates that there was a significantly enhanced percentage of HERV-K specific lysis of tumor cells, but not of uninvolved cells (P=0.043). In addition, there was a significantly enhanced percentage of HERV-K specific lysis of tumor cells, but not of benign disease cells (P=0.013). There was also a significantly enhanced percentage of HERV-K specific lysis of tumor cells, but not of benign cells in T cells depleted of Treg cells (P=0.006). A summary of CTL data is shown in FIGS. 4A-C.

Higher percentages of HERV-K specific lysis were observed in patients with ovarian cancer, compared to patients with benign diseases (P<0.0001) (FIG. 4A). Importantly, HERV-K was specific for killing target tumor cells, but not matched uninvolved normal epithelial cells (P=0.0006; FIG. 4B). In addition, enhanced HERV-K specific killing of tumor cells was observed if Treg cells were depleted (FIG. 4C). These findings have been demonstrated for the first time in preclinical trials. In conclusion, these data provide direct evidence that HERV-K env proteins are immunogenic in ovarian cancer patients.

Importantly, these studies are the first to directly show that these viral antigens induce T-cells and CTLs capable of killing HERV-K-expressing target cells, but not autologous uninvolved normal cells or benign cells obtained from patients without cancer. The re-activation of endogenous retroviral gene products and synthesis of mature protein products in cancer makes these a potentially valuable new pool of TAA for targeting of therapeutic vaccines to ovarian cancer.

Designing a Peptide Library and Identifying Specific Epitopes for B or T Cells.

Pools of overlapping peptides corresponding to specific antigens are frequently used to identify T cell immune response to vaccines or pathogens. A peptide library was used to assess both B cell and T cell response without the need to determine the patient's haplotype in advance. A study supporting this concept revealed that the level of Ab response against one Nef epitope of HIV-1 was correlated with HIV-1 disease progression (Yamada et al., 2004). Peptides of nine amino acids containing putative binding motifs for HLA-A0201 molecules have been selected for HERV-K env protein using the SYFPEITHI algorithm. 33 peptides (pool #1-12, Pool #12-23, and pool #24-33) have been synthesized and were tested in HLA-A2 normal donors or patients. A HERV-K env peptide overlap library composed of 144 15-mer peptides overlapping by 11 amino acids was prepared. The peptide library was used to assess both B cell and T cell response.

Identifying Specific Epitopes for B Cells.

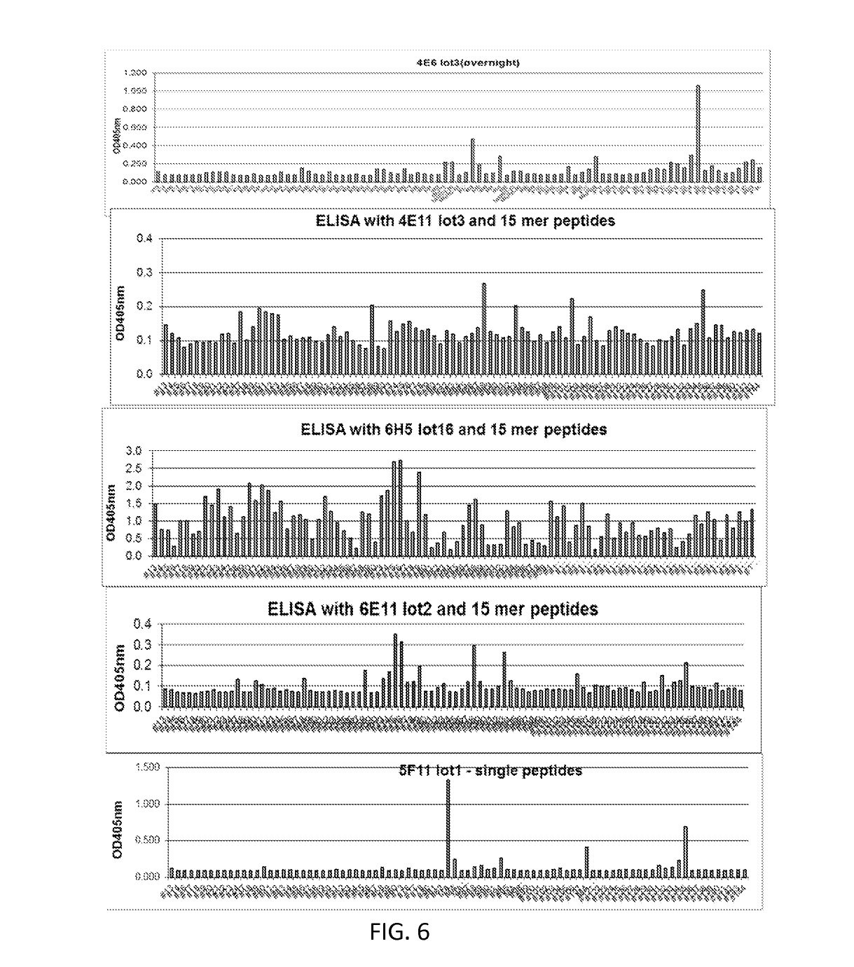

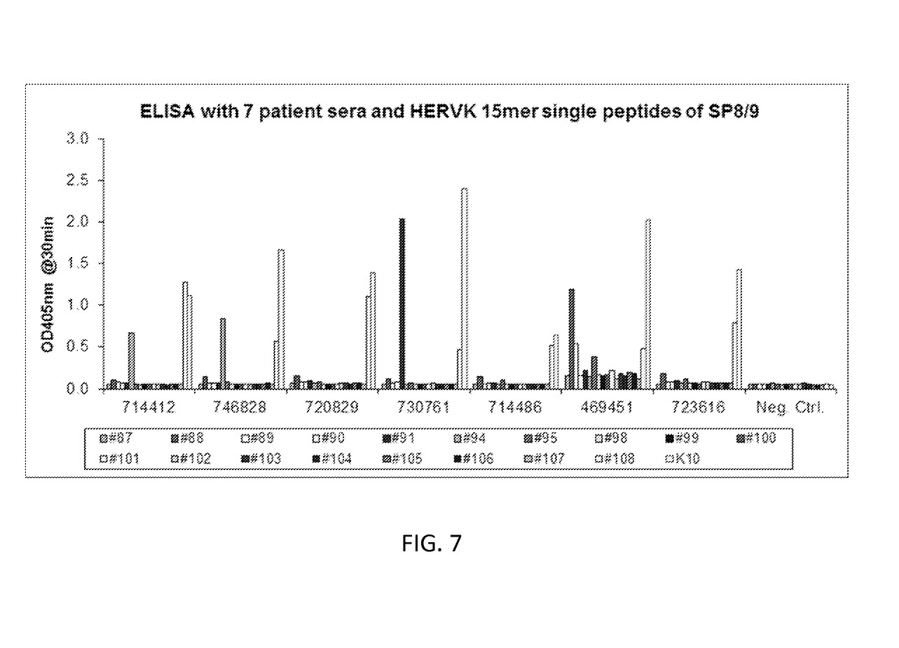

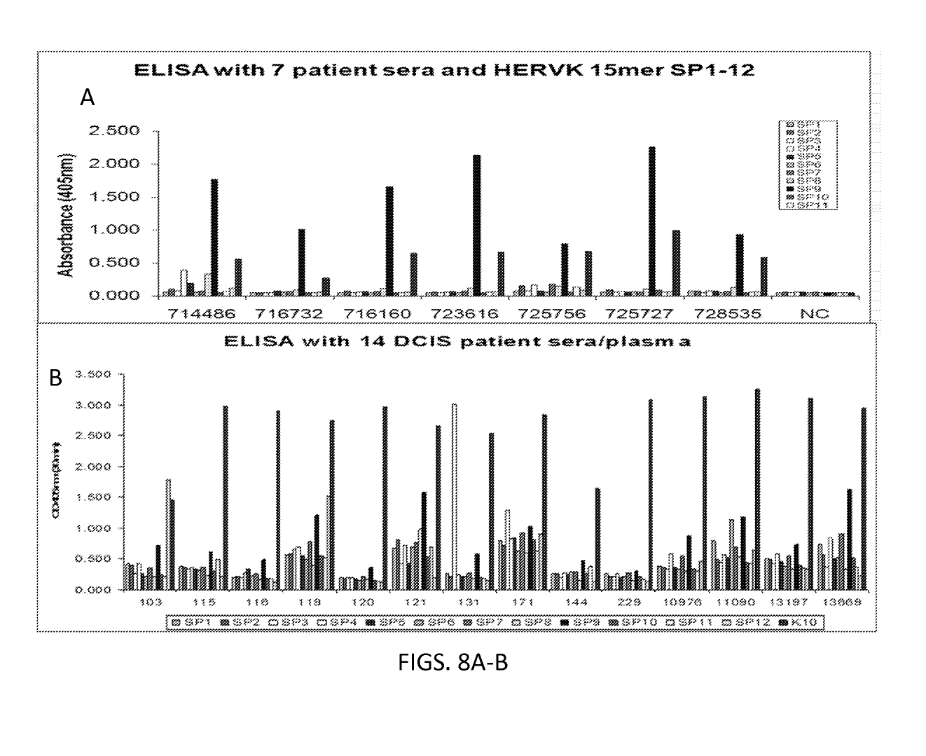

B cell epitopes have been screened by ELISA or flow cytometry using 4D1, 4E6, 4E11, 6H5, 6E11, 5F11mAbs or G11D10 or P 1/P2e (single chain antibodies generated from 6H5 (FIGS. 5-6), or the sera obtained from cancer patients (FIGS. 7-8). Several anti-HERV-K monoclonal antibodies were able to recognize some of these peptides.

From FIG. 5, monoclonal antibodies (4E6 and 4E11) detected B epitopes in SP7, SP8, and SP9. 6H5 detected B epitopes in SP2, SP 4, SP 5, SP 7, SP8, SP11, and SP12. Furthermore, single peptides were used for B-epitope detection (FIG. 6). Multiple peptides were detected with 6H5 mAb. Several epitopes were found by ELISA in SP8 or SP9 using patient sera (FIG. 7), such as 94, 108, 91, 88, 89, and 95. Patient sera were employed to determine B epitopes (FIG. 8) and B epitopes were detected in most of cancer patients in SP9.

Biacore Epitope Mapping.

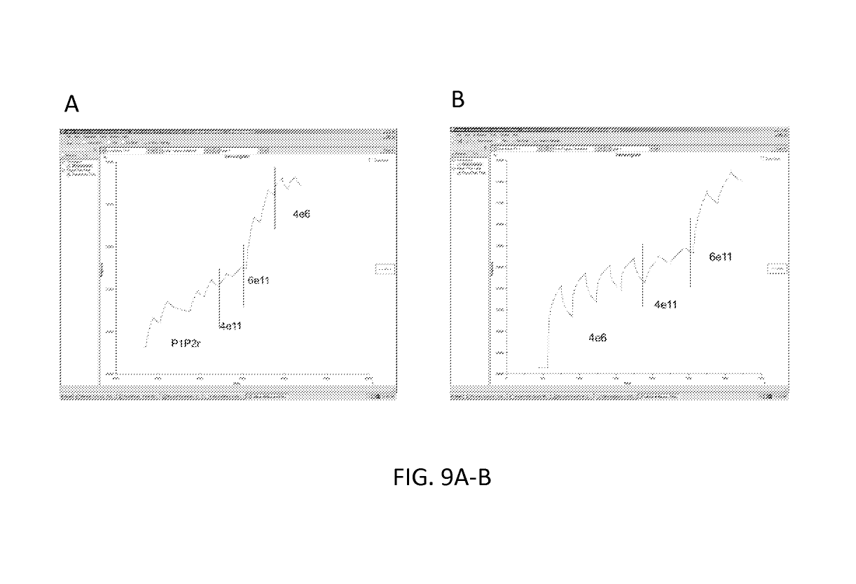

Biacore epitope mapping was employed to determine the overlap of epitopes among different monoclonal antibodies (FIGS. 9A-B). A CMS chip had 200 RU K10 g ligand immobilized on one flow cell (test) and another flow cell was activated and quenched with no ligand (control). 10 μg/ml of each antibody (analyte) was flowed over the chip surface in the order indicated in the sensorgram, with no regeneration between shots. Each antibody was injected multiple times to ensure epitope saturation. Binding of successive analytes is indicated by the development of a distinct secondary association curve.

The above data were matched with ELISA data (FIG. 6). The major binding epitopes are positions 88 and 135 for 4E6 and 4E11. In contrast, the major binding epitopes are positions 75 to 76 for 6H5 or 6E11. Also, 4E6 and 4E11 mAbs are IgG1a and 6H5 and 6E11 are IgG2a.

Development of a Peptide-Based Vaccine for Cancer Treatment Using the Multiple Antigen Peptide System (MAPS).

After identifying specific B cell epitopes, interest focused on developing a peptide-based cancer vaccine with the multiple antigen peptides (MAPs) system, and studying the immunogenicity of the vaccine. The MAP system consists of a large number of the same or different synthetic peptides bound to the groups of a dendritic core molecule providing a high concentration of antigen in a low molecular volume. The MAP system is a valuable approach for eliciting immune responses to peptides and developing therapeutic vaccines.

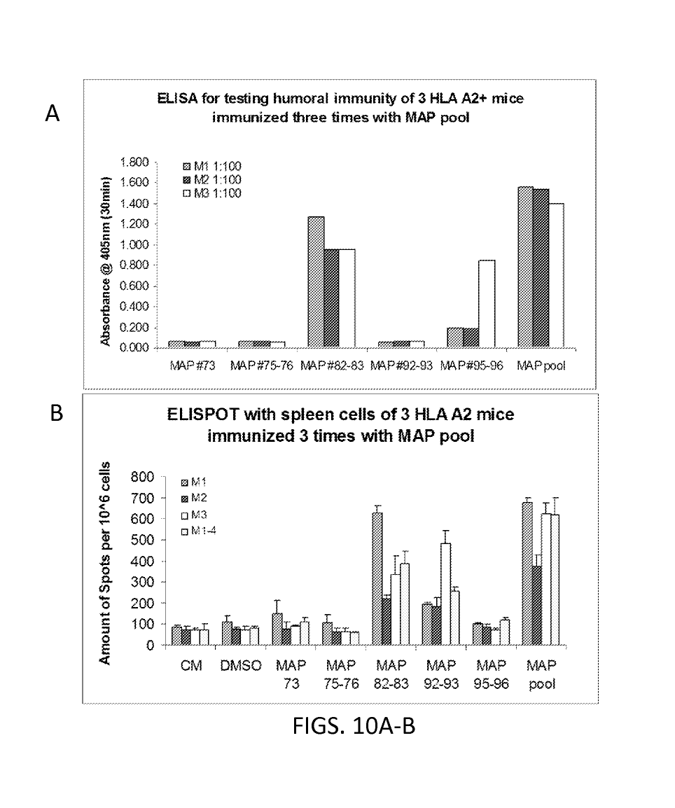

Candidate MAPs have been synthesized and tested in a mouse model. In this model, Balb/c mice were immunized subcutaneously 3 times with the MAP system containing the candidate epitopes, according to the immunization protocol. The immunized mice were sacrificed 10-14 days after the last boost and spleen cells and blood were harvested. Anti-mouse IFN-γ ELISPOT and ELISA assays were conducted to test both cellular and humoral immune responses against the candidate epitopes with mouse spleen cells and sera (FIGS. 10A-B).

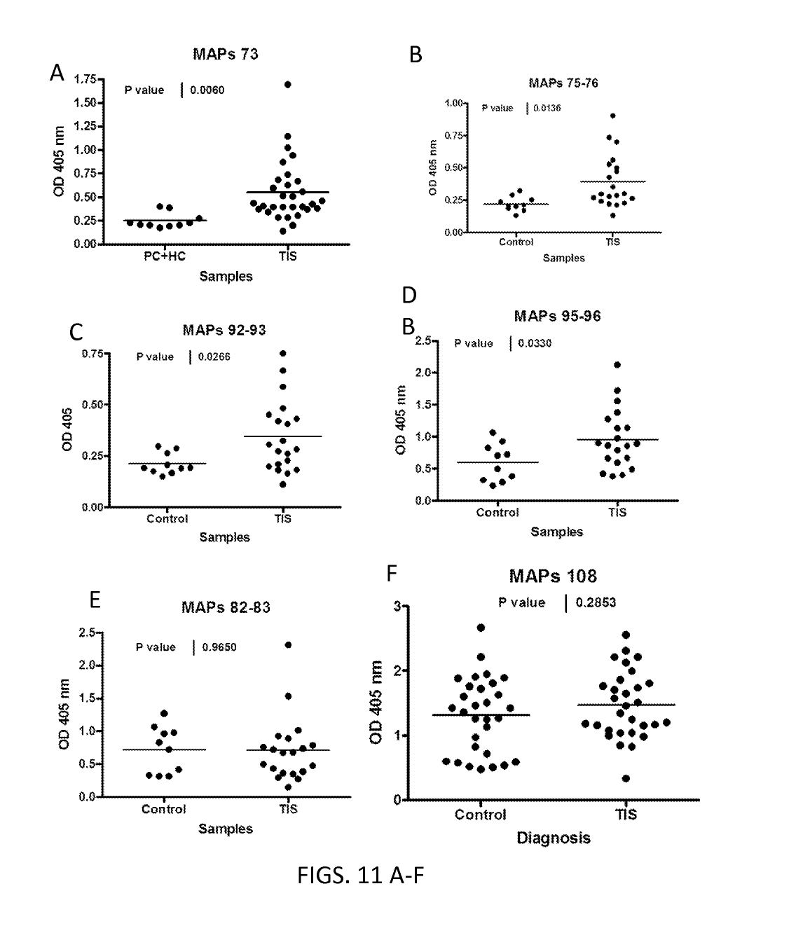

The presence of antibodies against the MAPS was tested in patient sera (FIGS. 11A-B). Significantly increased titers of antibodies against MAPs 73, 75-76, 92-93, and 95-96 were observed in women with early breast cancer (TIS), but titers against MAPs 82-83 and MAPs 108 were not elevated only in only TIS patient sera, compared to controls (N=20). However, MAP 82-83 was identified as being active using the murine system (FIGS. 10A-B).

Investigation of HERV-K T-Cell Epitopes.

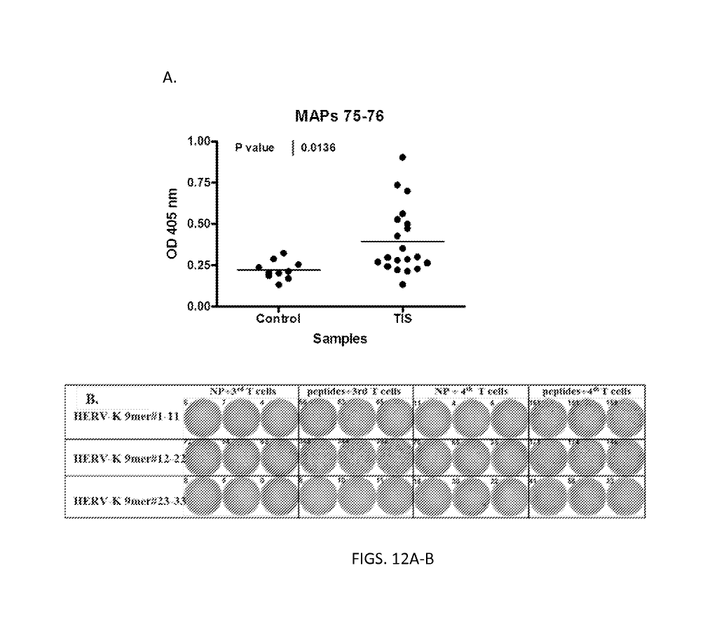

HLA-2+ patients were identified by flow cytometry using anti-A2 antibody. Activated B cells were cultured from PBMCs stimulated with irradiated 3T3-CD40L-expressing fibroblasts. Normal donor CD8+ T-cell lines were obtained from PBMCs stimulated with autologous activated B cells pulsed with peptide pools (peptides are in 3 pools: #1-11, #12-22, and #23-33); the stimulation was repeated for 3-4 cycles. HERV-K specific T cells generated from normal donor lines were tested for peptide reactivity using IFN-γ ELISPOT assays (FIGS. 12A-B).

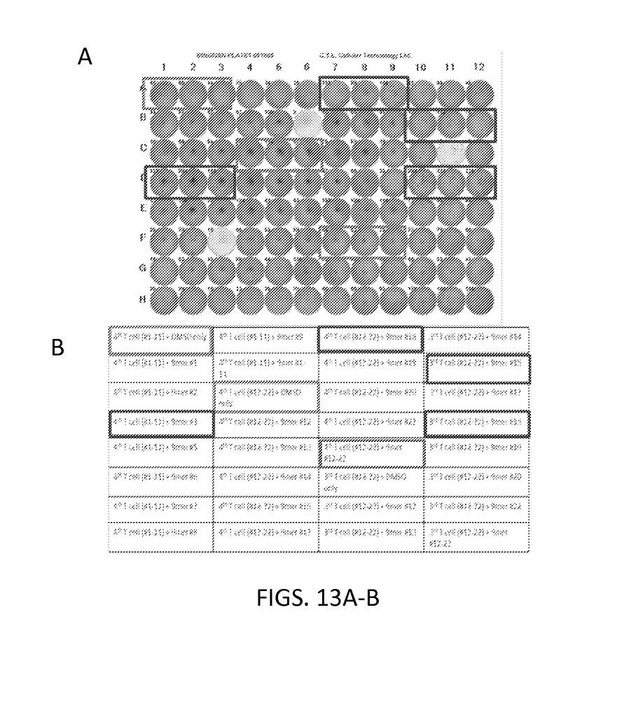

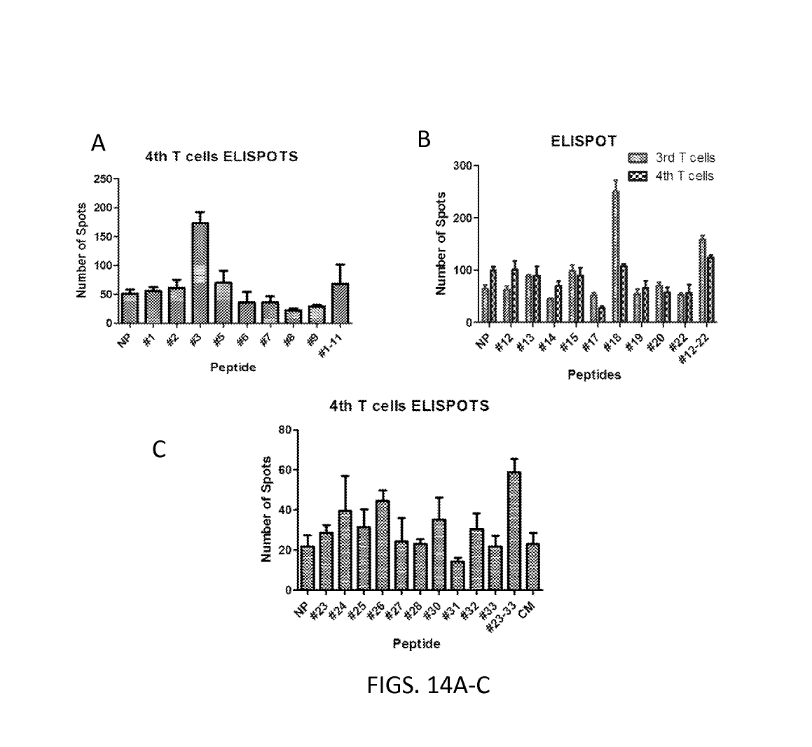

FIGS. 12A-B shows that T-cell epitopes were discovered in peptide pools 1-11 and 12-22, but not in 23-33 (Table 1). Furthermore, reactivity with individual peptides from within the pools was evaluated by ELISPOT (FIGS. 13A-B). T cell stimulated 4 times with peptide pool #1 to 11 produced numerous spots when reactived with Peptide #3 (FIG. 13A; raw; 1, 2, and 3). T cell stimulated 4 times with peptide pool #12 to 22 produced numerous spots when reactived with Peptide #18 (FIG. 13A; raw: 7, 8, and 9). T cell stimulated 3 times with peptide pool #12 to 22 produced numerous spots when reactived with Peptide #15 (FIG. 13B; raw: 10, 11, and 12). T cell stimulated 3 times with peptide pool #12 to 22 produced numerous spots when reactived with Peptide #18 (FIG. 13B; raw: 10, 11, and 12). A summary of the number of ELISPOTs from each individual peptide is shown in FIGS. 14A-C.

This research has identified two pools (#1 to 11 and #12 to 22; FIGS. 13A-B and 14A-C) from the HERV-K env 9-mer peptide library that contain potential CD8⁺ epitopes, using IFN-γ ELISPOT assay with T cells from HLA A2⁺ normal donors, which were stimulated with the 9-mer peptide pool-pulsed homologous B cells. CD8⁺ specific T epitopes were detected in 9-mer pools #1-11 and #12-22. Few spots were found in pools #23-33. Peptides 3 and 18 may be CD8 T epitopes for HLA-A2 donors,

Identification HERV-K Specific CD4 T Cells Epitopes.

IVS were obtained from two donor pairs stimulated with DC pulsed once with HERV-K protein. After 7 days (1-week stimulation), IVS cells were used for ELISPOT with 15-mer peptides (FIGS. 15A-B) or MAPs including MAP73, MAP75-76, MAP82-93, MAP 92-93, and MAP95-96 or 9 mer peptide pools (#1 to 5, #5 to 10, #1 to 10, #12 to 17, #18 to 22) or single peptides (#3, #18, and #4.11; FIG. 15C). Candidate epitopes (#4 and #11) were further identified using IVS generated from DC obtained from cancer patients stimulated with HERV-K antigen in this study.

From FIG. 15C, MAP73, MAP 92-93, 9-mer 3, 18, 4, and 11 from a donor (449731) are HERV-K specific T cell epitopes that will be further characterized in future studies. The T epitope peptide #73 (from SP7; Table 2) was further confirmed by ELISPOT using TIL obtained from melanoma patients (FIG. 16). As shown in FIG. 16, T epitopes (SP7; blue box) were recognized by TILs obtained from a melanoma patient. Furthermore, SP7 peptide #73 was the single peptide in SP7 which was recognized by TILs obtained from the melanoma patient. Additional TIL from various melanoma patients will be tested and used to determine which T epitopes are recognized by TIL from these patients.

In summary, the data provide strong evidence that HERV-K specific T and B cell epitopes can be identified by IFN′ ELISPOT and ELISA assays using in vitro stimulated cells and sera obtained from cancer patients.

Identification of HERV-K Epitopes Recognized by CD8⁺ T Cells.

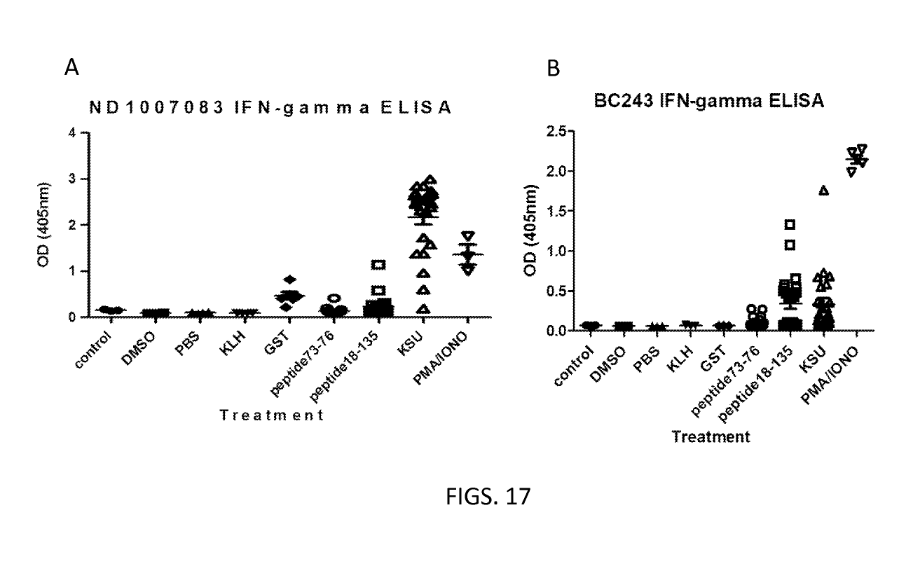

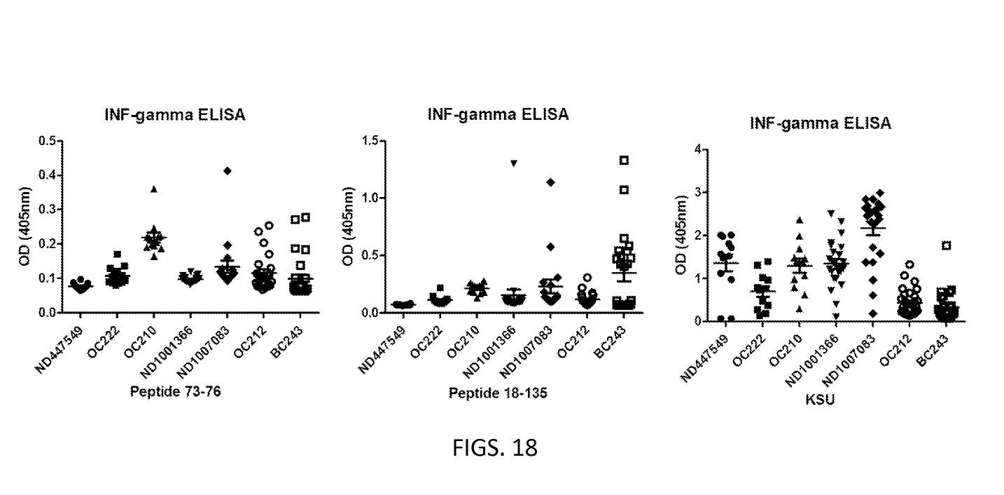

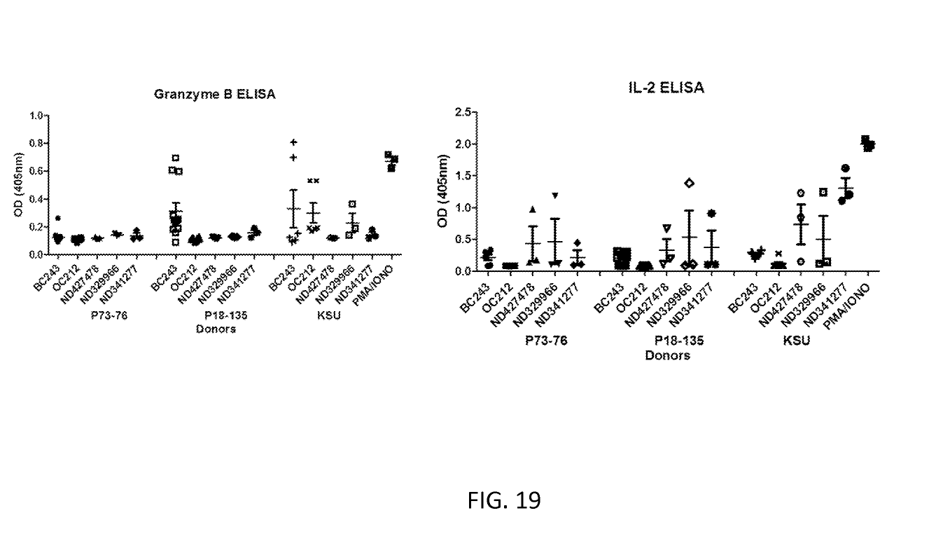

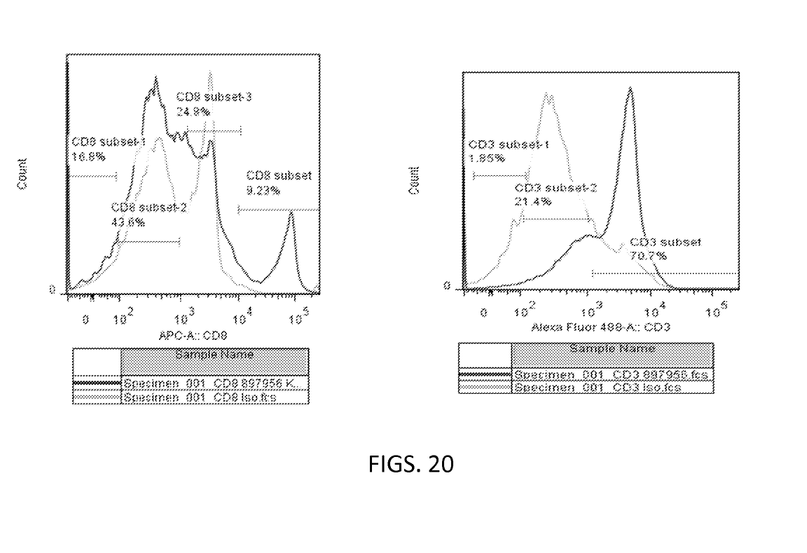

HERV-K-derived T cell epitopes recognized by CD8⁺ T cells were determined for cancer immunotherapy applications. The inventor selected two HERV-K peptides (Peptide #18-135 and Peptide #73-76) and HERV-K env SU protein, which was previously demonstrated to be antigenic. The two peptides were synthesized by Peptide 2 Inc., and along with HERV-K env SU protein, were evaluated in vitro for their ability to stimulate T cells in PBMCs from healthy subjects and/or cancer patients, based on interferon-γ (IFN-γ) release (FIGS. 17 and 18). One of these peptides, HERV-K₁₈₋₁₃₅, (as well as HERV-K SU protein) was found to induce IFN-γ release in peripheral T cells from both healthy subjects (ND1007083; FIG. 17, left) and cancer patients (BC 243; FIG. 17, right). HERV-K₇₃₋₇₆ was found to induce IFN-γ release in peripheral T cells from most cancer patients (FIG. 18, left; OC222, OC210, OC212, and BC 243). Importantly, Peptide #18-135 and Peptide #73-76 were found to induce granzyme B release in a breast cancer patient (BC243) to a greater extent than in 3 normal donors (FIG. 19, top). HERV-K env SU protein was found to induce granzyme B release in both cancer patients (BC243 and OC212) to a greater extent than in 3 normal donors. In contrast, Peptide #18-135, Peptide #73-76, and HERV-K env SU protein were found to induce IL-2 release in normal donors to a greater extent than in cancer patients. CD3+ and CD8+ T cells obtained from PBMCs of patient OC213 were pulsed with HERV-K SU protein, and two weeks later were sorted by flow cytometry (FIG. 20). Individual CD3+ and CD8+ T cells were used for further T expansion. Both CD3+ and CD8+ T will be used for further expansion. Expansion of T cells will be further used to recognize and kill HERV-K-expressing tumor cells. These results demonstrate the validity of the immuno-bioinformatics approach and suggest that HERV-K epitopes recognized by CD8+ T cells (especially HERV-K₁₈₋₁₃₅) may represent a new tumor-specific epitope for cancer immunotherapy.

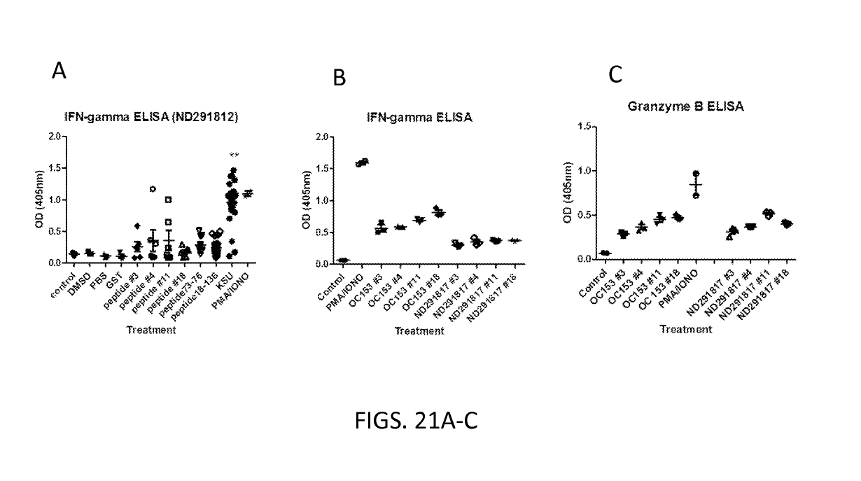

The inventor further investigated several 9-mer HLA-A2 peptides (HERVK-3, HERVK-4, HERVK-11, and HERVK-1; Table 1) in breast cancer, ovarian cancer, and normal female donors. OD values of IFN-γ release in these patients were compared to that in a normal donor (ND291812; FIG. 21A). Higher IFN-γ release was demonstrated in PBMC stimulated with HERVK-3, HERVK-4, HERVK-11, HERVK-18, p73-76, and p18-135 as well as HERV-K SU protein. Furthermore, IFN-gamma or Granzyme B release was compared in PBMC obtained from a cancer patient (OC153) and a normal donor (ND291817; FIGS. 21B-C). Higher IFN-γ release was demonstrated in PBMC obtained from OC153 than from a normal female donor.

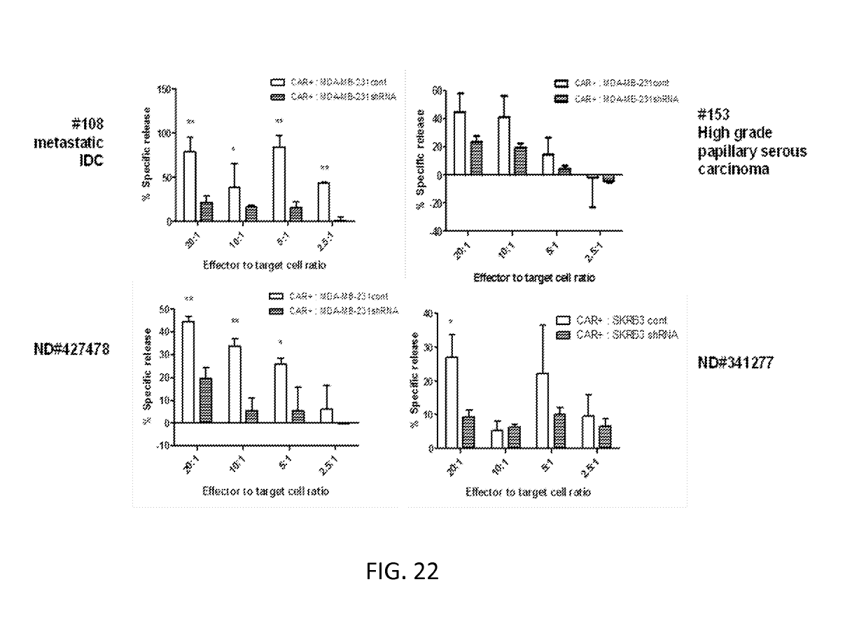

In order to determine HERV-K specific T cell killing in an antigen specific manner, the inventor employed shRNA to knock down the expression of HERV-K in target cells. Knockdown of HERV-K env RNA was achieved in breast cancer cells using shRNA targeting HERV-K env SU RNA (shRNA667). A transduced matched scrambled shRNA control (667c) did not knock down HERV-K in these cancer cells. A CTL assay was employed to determine HERV-K-specific chimeric antigen receptor (CAR) T cell cytotoxicity toward cancer cells. The scFv sequence of the HERV-K monoclonal antibody (6H5) was used to construct the CAR in Dr. Laurence Cooper's laboratory at MD Anderson Cancer Center. PBMCs obtained from BC108 (metastatic IDC), OC153 (high grade papillary serous carcinoma), and two normal donors were electroporated with HERV-K-CAR, then propagated on artificial antigen presenting (aAPC) cells (HERV-K+ K562 cells) in the presence of IL-2 and IL-21. Higher lysis was demonstrated in breast cancer cell lines transduced with control shRNA, compared with breast cancer cell lines transduced with HERV-K shRNA (FIG. 22). The data thus demonstrate that there was specific killing in an antigen-specific manner.

### Example 3

### Discussion

New antigens for immune therapy hold a promise for more effective, less toxic, and longer lasting disease control. One of the main hurdles encountered is the need to overcome self-tolerance mechanisms that limit the immune response against these types of tumor antigens. Viral antigens may be better suited to trigger stronger anti-tumor T-cell responses due to their foreign nature. Therefore, identification of viral T cell and B cell specific peptides in ovarian cancer antigens is important for the development of immunotherapies.

One group of viruses that has emerged recently to be associated with cancer is human retroviruses. Human endogenous retroviruses (HERVs), comprising 8.3% of the human genome, are silenced under normal conditions. Among the 30-50 families of HERVs, type K (HERV-K) contains functional full-length open reading frames (ORFs) encoding retroviral gag, pol and env proteins, which confers upon HERV-K the most biological activity. Previous research from the inventor's laboratory demonstrated that HERV-K envelope (env) and other HERV families are frequently expressed in human ovarian cancer, but not in normal ovarian tissue and uninvolved epithelial cells, at the mRNA and protein levels. Importantly, both humoral and cellular immune responses against HERV-K env protein were demonstrated in ovarian cancer patients but not in normal donors in the inventor's laboratory. The inventor proposes to identify HERV-K env antigen-derived peptides recognized by CD8⁺ T, CD4⁺ T and B cells in breast cancer and/or ovarian cancer patients or other cancer patients, and to test the effects of anti-HERV antigen vaccination in these cancer patients.

The human genome harbors many ERV sequences and some of them may continue to perform retroviral functions, including tumor induction. The inventor demonstrated HERV-K specific T cell cytotoxicity toward only ovarian tumor cells and not normal ovarian cells. Increased secretion of INF-γ and TNF-α was confirmed in HERV-K specific T cells. Previous studies have shown that these cytokines, especially IFN-γ and TNF-α (Th1), are very important in promoting cancer patient survival.

The re-activation of endogenous retroviral gene products and synthesis of mature protein products in cancer makes these a potentially valuable new pool of antigens for targeting of therapeutic vaccines for ovarian cancer. The silencing of retroviral gene expression throughout one's lifetime and its reactivation specifically in ovarian cancer cells suggests that immunological self-tolerance mechanisms against HERV-K and other retroviral proteins may be limited or that the immune system exists in a state of ignorance towards these antigens until cancer develops. The specific re-activation of retroviral protein expression in ovarian cancer cells raises the exciting possibility of prophylactic vaccination to prevent primary ovarian tumor development. Similar to anti-viral vaccines like anti-HPV vaccines now used to prevent cervical cancer, prophylactic vaccines against hitherto non-expressed retroviral antigens may elicit long-lived retroviral antigen-specific T cell responses (T-central memory) in an otherwise ignorant host that can eradicate early malignancies re-expressing these retroviral gene products. These results provide further evidence that HERVs are a previously overlooked group of tumor-specific antigens expressed in ovarian cancer patients. They further provide valuable information on the potential of this immunotherapeutic regimen against HERV-K positive ovarian cancer and on the potential to dramatically improve the lives of patients suffering from ovarian cancer.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
U.S. Pat. No. 4,435,386
U.S. Pat. No. 4,436,727
U.S. Pat. No. 4,436,728
U.S. Pat. No. 4,505,899
U.S. Pat. No. 4,505,900
U.S. Pat. No. 4,520,019
U.S. Pat. No. 4,554,101
U.S. Pat. No. 4,579,945
U.S. Pat. No. 4,684,611
U.S. Pat. No. 4,797,368
U.S. Pat. No. 4,866,034
U.S. Pat. No. 4,877,611
U.S. Pat. No. 4,950,645
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,656,610
U.S. Pat. No. 5,702,932
U.S. Pat. No. 5,736,524
U.S. Pat. No. 5,739,169
U.S. Pat. No. 5,780,448
U.S. Pat. No. 5,789,215
U.S. Pat. No. 5,801,005
U.S. Pat. No. 5,824,311
U.S. Pat. No. 5,830,880
U.S. Pat. No. 5,846,945
U.S. Pat. No. 5,945,100
U.S. Pat. No. 5,994,136
U.S. Pat. No. 5,994,624
U.S. Pat. No. 6,013,516
Austin-Ward and Villaseca, Rev. Med. Chil., 126(7):838-45, 1998.
Azuma et al., Cell Immunol., 116(1):123-134, 1988.
Baichwal and Sugden, In: Gene Transfer, Kucherlapati (Ed.), NY, Plenum Press, 117-148, 1986.
Bajorin et al., J. Clin. Oncol., 6(5):786-92, 1988.
Benjamini et al., Adv. Exp. Med. Biol., 303:71-77, 1991.
Blomer et al., J. Virol., 71(9):6641-6649, 1997.
Brutlag et al., Comput. Appl. Biosci., 6(3):237-245, 1990.
Bukowski et al., Clin. Cancer Res., 4(10):2337-47, 1998.
Capaldi et al., Biochem. Biophys. Res. Comm., 74(2):425-433, 1977.
Chen and Okayama, Mol. Cell Biol., 7(8):2745-2752, 1987.
Chou and Fasman, Annu. Rev. Biochem., 47:251-276, 1978a.
Chou and Fasman, Biochemistry, 13(2):211-222, 1974b.
Chou and Fasman, Biophys. J., 26(3):385-399, 1979.
Christodoulides et al., Microbiology, 144(Pt 11):3027-37, 1998.
Cotten et al., Proc. Natl. Acad. Sci. USA, 89(13):6094-6098, 1992.
Coupar et al., Gene, 68:1-10, 1988.
Culver et al., Science, 256(5063):1550-1552, 1992.
Curiel, Nat. Immun., 13(2-3):141-164, 1994.
Davidson et al., J. Immunother., 21(5):389-98, 1998.
Dillman, Cancer Biother. Radiopharm., 14(1):5-10, 1999.
Fechheimer et al., Proc. Natl. Acad. Sci. USA, 84:8463-8467, 1987.
Fetrow and Bryant, Biotechnology, 11(4):479-484, 1993.
Fraley et al., Proc. Natl. Acad. Sci. USA, 76:3348-3352, 1979.
Friedmann, Science, 244:1275-1281, 1989.
Gopal, Mol. Cell Biol., 5:1188-1190, 1985.
Graham and Van Der Eb, Virology, 52:456-467, 1973.
Grunhaus and Horwitz, Seminar in Virology, 3:237-252, 1992.
Hanibuchi et al., Int. J. Cancer, 78(4):480-485, 1998.
Harlan and Weintraub, J. Cell Biol., 101:1094-1099, 1985.
Hellstrand et al., Acta Oncol., 37(4):347-353, 1998.
Horwich et al. J. Virol., 64:642-650, 1990.
Hui and Hashimoto, Infect. Immun., 66(11):5329-36, 1998.
Husson et al., J. Bacteriol., 172(2):519-524, 1990.
Irie and Morton, Proc. Natl. Acad. Sci. USA, 83(22):8694-8698, 1986.
Irie et al., Lancet., 1(8641):786-787, 1989.
Jacobs et al., Nature, 327(6122):532-535, 1987.
Jameson and Wolf, Comput. Appl. Biosci., 4(1):181-186, 1988.
Johnson et al., In: Biotechnology And Pharmacy, Pezzuto et al. (Eds.), Chapman and Hall, NY, 1993.
Ju et al., J. Neuropathol. Exp. Neurol., 59(3):241-50, 2000.
Kaeppler et al., Plant Cell Reports, 9:415-418, 1990.
Kaneda et al., Science, 243:375-378, 1989.
Kato et al, J. Biol. Chem., 266:3361-3364, 1991.
Kelleher and Vos, Biotechniques, 17(6):1110-7, 1994.
Kyte and Doolittle, J. Mol. Biol., 57(1):105-32, 1982.
Laughlin et al., J. Virol., 60(2):515-524, 1986.
Lebkowski et al., Mol. Cell. Biol., 8(10):3988-3996, 1988.
Lotte et al., Adv. Tuberc. Res., 21:107-93; 194-245, 1984.
Luelmo, Am. Rev. Respir. Dis., 125(3 Pt 2):70-72, 1982.
Mann et al., Cell, 33:153-159, 1983.
Martin et al., Nature, 345(6277):739-743, 1990.
McLaughlin et al., J. Virol., 62(6):1963-1973, 1988.
Miller et al., Am. J. Clin. Oncol., 15(3):216-221, 1992.
Muzyczka, Curr. Topics Microbiol. Immunol., 158:97-129, 1992.
Naldini et al., Science, 272(5259):263-267, 1996.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Omirulleh et al., Plant Mol. Biol., 21(3):415-28, 1993.
Paskind et al., Virology, 67:242-248, 1975.
Paul, Transplant Proc., 25(2):2080-2081, 1993.
PCT Appln, WO 91/16347
PCT Appln. WO 94/09699
PCT Appln. WO 95/06128
Pietras et al., Oncogene, 17(17):2235-49, 1998.
Porter-Jordan & Lippman, Breast Cancer 8:73-100, 1994.
Potrykus et al., Mol. Gen. Genet., 199:183-188, 1985.
Qin et al., Proc. Natl. Acad. Sci. USA, 95(24):14411-14416, 1998.
Rabinovich et al., Science, 265(5177):1401-1404, 1994.
Remington's Pharmaceutical Sciences, 15^{th} ed., pages 1035-1038 and 1570-1580, Mack Publishing Company, Easton, Pa., 1980.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Rosenberg et al., Ann. Surg. 210(4):474-548, 1989.
Rosenberg et al., N. Engl. J. Med., 319:1676, 1988.
Roux et al., Proc. Natl. Acad. Sci. USA, 86:9079-9083, 1989.
Snapper et al., Proc. Natl. Acad. Sci. USA, 85(18):6987-6991, 1988.
Takada et al., J. Clin. Microbiol., 33(3):658-660, 1995.
Temin, In: Gene Transfer, Kucherlapati (Ed.), NY, Plenum Press, 149-188, 1986.
Tratschin et al., Mol. Cell. Biol., 4:2072-2081, 1984.
Wada et al., Nucleic Acids Res. 18:2367-2411, 1990.
Wang-Johanning et al., Cancer Res 68(14): 5869-77 2008.
Weinberger et al., Science, 228:740-742, 1985.
Wolf et al., Comput. Appl. Biosci., 4(1):187-191, 1988.
Wong et al., Gene, 10:87-94, 1980.
Yamada et al., J. Immunol. 172(4): 2401-6, 2004.
Yamamoto et al., Jpn. J. Cancer Res., 79:866-873, 1988.
Yin et al., J. Biol. Resp. Modif., 8:190-205, 1989.
Zufferey et al., Nat. Biotechnol., 15(9):871-875, 1997.

1. A vaccine comprising a HERV-K enveloped (env) peptide or polypeptide comprising a sequence selected from SEQ ID NO: 1-179.

2. The vaccine of claim 1, wherein the peptide or polypeptide comprises 9 to 50 consecutive residues of HERV-K env.

3. (canceled)

4. The vaccine of claim 1, wherein the peptide is less than 50 residues in length.

5. (canceled)

6. The vaccine of claim 1, further comprising an adjuvant.

7. (canceled)

8. The vaccine of claim 1, further comprising an immunostimulatory cytokine.

9. (canceled)

10. The vaccine of claim 1, wherein the peptide or polypeptide is selected based on the antigenic profile of tumor to be treated or based on the HLA type of the patient.

11. (canceled)

12. The vaccine of claim 1, wherein said peptide or polypeptide comprises a T cell epitope.

13-14. (canceled)

15. The vaccine of claim 1, wherein said peptide or polypeptide comprises a B cell epitope.

16. A method for treating a cancer in a patient comprising administering to said patient a therapeutically effective amount of a vaccine comprising HERV-K enveloped (env) peptide or polypeptide comprising a sequence selected from SEQ ID NO: 1-179.

17-19. (canceled)

20. The method of claim 16, wherein said cancer is a solid tumor.

21-22. (canceled)

23. The method of claim 22, wherein the peptide or polypeptide comprises 9 to 50 consecutive residues of HERV-K env.

24. (canceled)

25. The method of claim 16, wherein the peptide is less than 50 residues in length.

26. (canceled)

27. The method of claim 16, wherein the vaccine is administered systemically or locally.

28-31. (canceled)

32. The method of claim 29, wherein the peptide or polypeptide comprises a T cell epitope.

33-34. (canceled)

35. The method of claim 16, wherein said peptide or polypeptide comprises a B cell epitope.

36. The method of claim 16, further comprising treating the patient with a second anticancer therapy.

37-40. (canceled)

41. A method for treating cancer in a patient comprising:
(a) contacting CTLs of said patient with a HERV-K enveloped (env) peptide or polypeptide comprising a sequence selected from SEQ ID NO:1-33, 178 and 179; and
(b) administering a therapeutically effective amount of the CTLs of step (b) to the patient.

42. The method of claim 41, further comprising expanding said CTL's by ex vivo or in vivo methods prior to administration.

43-45. (canceled)

46. A method for treating a cancer in a patient comprising administering to said patient a therapeutically effective amount of a vaccine comprising an expression construct encoding a HERV-K enveloped (env) peptide or polypeptide comprising a sequence selected from SEQ ID NO: 1-179.

47-48. (canceled)

49. The method of claim 46, wherein said expression construct is located in an antigen presenting cell.

50. (canceled)

51. A method of identifying a subject with breast and ovarian cancer comprising determining the presence or amount of an anti-HERV-K envelope protein immune response in a sample from said subject.

52. The method of claim 51, wherein said immune response is a humoral immune response.

53. (canceled)

54. The method of claim 51, wherein said immune response is a cellular immune response.

55-56. (canceled)

57. The method of claim 51, wherein said sample is peripheral blood, serum, plasma, or a tumor biopsy.

58. The method of claim 51, further comprising assessing the presence or amount of an anti-HERV-K envelope protein immune response in a sample from said subject a second time.

59. The method of claim 58, further comprising positively correlating a reduced immune response over time with therapeutic response.

60. The method of claim 58, further comprising treating the patient to produce a therapeutic response.

61. A method for stimulating an anti-cancer B cell response in a cancer patient comprising:
(a) contacting B cells of said patient with a HERV-K enveloped (env) peptide or polypeptide comprising a sequence selected from SEQ ID NO:34-177; and
(b) administering a therapeutically effective amount of the B cells of step (a) to the patient.

62. The method of claim 61, further comprising stimulating said B cells ex vivo or in vivo methods prior to administration.

63-65. (canceled)
